(19) United States
(12) Patent Application Publication
(10) Pub. No.: US 2005/0143321 A1
(43) Pub. Date: Jun. 30, 2005

(54) Composition with stabilized redox properties and method of stabilization of redox properties

(75) Inventors: Irna Viktorovna Vardosanidze, Moscow (RU); Vitality Evgenievich Pilkin, Moscow (RU); Valdimir Mironovich Dvornikov, Moscow (RU)

Correspondence Address: MORRISON & FOERSTER LLP, 1650 TYSONS BOULEVARD, SUITE 300, MCLEAN, VA, 22102, US

(73) Assignee: Irina Viktorovna VARDOSANIDZE Vitality Evgenievich PILKIN, Moscow (RU)

(21) Appl. No.: 10/500,301

(22) PCT Filed: Jul. 23, 2003
(86) PCT No.: PCT/RU03/00328

(30) Foreign Application Priority Data
Oct. 15, 2002 (RU) 2002127470

(51) Int. Cl. A61K 7/06; A61K 7/11; A61K 38/39

(52) U.S. Cl. 514/21; 424/70.14

(57) ABSTRACT

There is offered the composition with stabilized redox properties, which corresponds to water solution and/or raw material-containing water with spontaneously changing redox properties, which are characterized by spontaneous increasing of the redox potential, in which redox properties are stabilized by addition of amino-acids with not charged polar substitutes and/or their derivatives and/or peptides, containing mentioned amino-acids and/or their derivatives and/or their mixtures, which could be used in food industry, medicine, veterinary, pharmaceutical industry, cosmetic industry, balneology, agriculture, fish farming and other fields.

### FIELD OF THE INVENTION

The invention relates to the field of physical chemistry, in particular, to chemistry of solutions, colloid chemistry and electrical chemistry, and also to nutritive chemistry, and used for stabilization of changing redox properties of water solutions and raw material-containing water, which are characterized by spontaneous increasing of the redox potential in relation to the potential of the hydrogen electrode, which value is considered as zero, and could be used in food industry, medicine, veterinary medicine, pharmaceutical industry, cosmetic industry, balneology, agriculture, fish farming and other branches of technics.

### BACKGROUND OF THE INVENTION

There are known water, water solutions and raw material-containing water, natural and artificial, with spontaneously changing redox properties, which are characterized by spontaneous increasing of the redox potential in relation to the potential of the hydrogen electrode, which value is considered as zero.

The natural water solutions and raw material-containing water with specified redox properties are, for example, saliva, blood, breast milk, hydrosulphuric, ferrous, nitric, hydrogen mineral waters, hydrosulphuric and other mud, sludge, peat.

Artificial waters, water solutions and raw material-containing water with specified redox properties are some alcoholic drinks, such as beer, some alcohol-free drinks, such as kvas, hydrosulphuric waters, and mud, water solutions with dissolved biologically active additive “Microhydrine”, water and water solutions, which obtain the specified redox properties in result of electrochemical (cathode) reduction on electrodes in electrolytic bath, for example STEL, “Izumrud” etc., as well as water and water solutions, which obtain the specified redox properties by any other way (see B. I. Leonov, V. I. Prilutsky, V. M. Bakhir—Physical and chemical aspects of biological effect of activated water, Moscow, 1999, p. 163, or, for example, patent RF N2155717 of 01.28.200, or, for example, V. G. Shironosov, O. A. Dubrovskaya, R. F. Mullakhmetov—Phenomenon of the contactless activation during electrolysis, due to microhydrine and during chemical reactions. Third International symposium “Electrochemical activation in medicine, agriculture and industry. Moscow, 2001, p. 48).

Artificial water solutions and raw material-containing water with specified redox properties are also the mixtures of any raw materials with water or water solution with specified redox properties.

In context of this invention we understand the electrochemically reduced water or electrochemically reduced water solution or cathode reduced water or cathode reduced water solution as water or water solutions, which are in unbalanced, i.e. in metastable condition under some physical and chemical influence, for example, electrical current. Such water solutions are described in literature by such terms as electrochemically activated water or catholyte or modified water etc. (see, for example, N. L. Glinka. General Chemistry. M. Integral-Press, 2002, p. 283, and also V. I. Prilutsky, V. M. Bakhir. Electrochemically activated water: anomalous properties, mechanism of biological effect, M. 1997, p.p. 4-5, and also, for example, E. E. Fesenko at al. Immunomodulating properties of bi-distillated modifies water. Biophysics, 2001, v. 45, issue 2, p. 353).

Under the spontaneous increasing of redox potential in context of this invention we understand the spontaneous increasing of the value of redox potential, which has mainly the negative value or the value close to the value of the hydrogen electrode, which potential is considered as zero, and depends on properties of elements and compositions to “give away or receive” electrons, and the measure of such properties is the affinity to electron (see G. E. Levant, G. A. Raitsin. Practical guide in general chemistry. M., Vysshaya shkola, 1971, p. 154-155, N. L. Glinka. General chemistry. M., Integral-Press, 2002, p. 82).

Under the redox potential in the context of this invention we understand the potential difference (e.m.f.—electromotive force) in an electrochemical circuit, which forms during immersion of any metal in the water solution of its salts, and which consists, for example, of the standard hydrogen electrode and, for example, of silver chloride electrode of comparison (SCE).

Redox potential in context of this invention is the measure of redox properties of mentioned solutions and raw material-containing water.

Redox potential characterizes the activity rate of electrons in redox reactions in mentioned solutions and raw material-containing water connected with affixture or transfer of electrons to oxidizer from reducer correspondingly.

The value of the redox potential is expressed in millivolts and could have both positive and negative value in relation to the standard hydrogen electrode, the value of which is considered as zero.

In reduction conditions the redox potential is negative, and it depends also on the value of hydrogen ion exponent (pH) of solutions, for example, in case of dissolution of oxygen, including atomic, or hydrogen sulphide etc. in water (see, for example, S. R. Krainov, V. M. Shvets. Hydrogeochemistry. M., Nedra, 1992. P. 129, and also, for example, Resort resources of USSR. Medgiz. 1956. p. 400-401).

SCE in context of this invention means that the measurement is executed by the platinum electrode (hydrogen) with the silver chloride electrode of comparison.

Water solution and raw material-containing water with spontaneously changing redox properties, which are characterized by spontaneous increasing of redox potential, in context of this invention have mainly negative potential or potential close to potential of the hydrogen electrode, which value is considered as zero; both in area of negative values and in area of positive values the redox potential has some useful properties, for example, disinfectant, antiseptic, protective, antioxidant, antiphlogistic, antimutagenous, radio-protective, immunostimulating, adoptogenous, virulicide, antiviral, regenerative, solvent, catalytic and other useful properties.

If protect mentioned water solution and raw material-containing water from oxidation, mechanical, radiation and other external influences, their biological activity and chemical reaction ability will disappear completely and spontaneously within the period from 15 minutes to 20 days, subject to degree of mineralization of initial water solution, with spontaneous increasing of redox potential from negative values to positive values in relation to the potential of the hydrogen electrode, which value is considered as zero.

It is known the method of 7-10 times increasing stability of low mineralized water solution of salts, in particular, of sodium chloride, with concentration up to 3-5 grams per litre, with spontaneously changing redox properties, which are characterized by spontaneous increasing of the redox potential in relation to the potential of the hydrogen electrode, which value is considered as zero, by increasing of ion force of the solution with initial concentration of sodium chloride in amount of 10⁻⁴ mole/litre of water to 0.1 mole/litre by addition of small volume of concentrated solution of sodium chloride (see V. I. Lobyshev, I. Y. Petrushanko (Popova), V. I. Kiselev. Electrochemical activation of water. In: Third international symposium. Moscow. 2001. p. 76).

Demerit of mentioned methods is the fact that the used methods lead to increasing of mineralization and significant shifts of acid-alkaline equilibrium of water, water solution and raw material-containing water, which is unacceptable for creation of foodstuff on the basis of methods, known now, as well as the fact that maximum within several days water solutions and raw material-containing water completely lose their different useful properties (see, for example, V. I. Prilutsky, V. M. Bakhir “Electrochemically activated water: anomalous properties, mechanism of biological effect, Moscow, 1997, pp. 64-66).

### SUMMARY OF THE INVENTION

The purpose of this invention is the stabilization of redox properties of composition corresponding to water, water solution and raw material-containing water with spontaneously changing redox properties, which are characterized by spontaneous increasing of the redox potential in relation to the potential of the hydrogen electrode, which value is considered as zero, for preservation of useful properties of mentioned composition.

Author of this invention all at once has found out that mentioned problem could be solved by addition in the composition corresponding to water, water solution and raw material-containing water with spontaneously changing redox properties, which are characterized by spontaneous increasing of the redox potential in relation to the potential of the hydrogen electrode, which value is considered as zero, of amino-acids with not charged polar substitutes in the structure of amino-acids, in which number there are glycine, serine, threonine, cysteine, tyrosine, asparagine, glutamine and/or their derivatives and/or peptides, containing mentioned amino-acids and/or their derivatives and/or their mixtures.

In accordance with the first aspect the invention allows to create composition with stabilized redox properties, which corresponds to water solution and/or raw material-containing water with spontaneously changing redox properties, which are characterized by spontaneous increasing of the redox potential in relation to the potential of the hydrogen electrode, which value is considered as zero, in which redox properties are stabilized by addition of amino-acids with not charged polar substitutes and/or their derivatives and/or peptides, containing mentioned amino-acids and/or their derivatives and/or their mixtures. Amino-acids with not charged polar substitutes could be presented by glycine, serine, threonine, cysteine, tyrosine, asparagine or glutamine. Peptides could be presented by gelatin. Volume of amino-acids with not charged polar substitutes and/or their derivatives and/or peptides containing mentioned amino-acids and/or their derivatives and/or their mixtures could be more than 0.005% of weight.

Composition with redox properties, stabilized according to this invention, could be a foodstuff, including mineral and/or drinking water, milk composition, juice, alcoholic and/or alcohol-free drink, mayonnaise, ketchup, sauce, meat, fish, vegetable and/or fruit semi-manufactured product, sausage or canned composition, confectionery, bread, macaroni, foodstuff acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be the balneotherapeutic composition, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive of vitamin mixture, liquid for contact lenses, eye drops, basis for medical preparation, influencing different kinds of metabolism, including carbohydrate metabolism, phosphoric and calcium metabolism, homeostasis, hemopoiesis, hemostasis; agent, influencing immunity, correcting antitumoural therapy, antibiotics therapy, radio therapy used in gynecology, otorhynolaryngology, dentistry, ophthalmology, proctology, urology, for external use, dermatology; agents with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be also the composition for cosmetic purpose, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be also the composition for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be also the composition for veterinary purposes, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Composition with redox properties, stabilized in accordance with the invention, could be also used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.

In accordance with the second aspect the invention gives the method of stabilization of redox properties of composition, which corresponds to water solution and/or raw material-containing water with spontaneously changing redox properties, which are characterized by spontaneous increasing of the redox potential in relation to the potential of the hydrogen electrode, which value is considered as zero, which consists in addition of amino-acids with not charged polar substitutes and/or their derivatives and/or peptides, containing mentioned amino-acids and/or their derivatives and/or their mixtures, in which mentioned amino-acids could be presented by glycine, serine, threonine, cysteine, tyrosine, asparagine or glutamine, and mentioned peptide could be presented by gelatin. Volume of amino-acids with not charged polar substitutes and/or their derivatives and/or peptides containing mentioned amino-acids and/or their derivatives and/or their mixtures could be more than 0.005% of weight.

The invented method could be used for stabilization of redox properties of water solutions and/or raw material-containing water, which could be presented by foodstuffs, including mineral and/or drinking water, milk composition, juice, alcoholic and/or alcohol-free drink, mayonnaise, ketchup, sauce, meat, fish, vegetable and/or fruit semi-manufactured product, sausage or canned composition, confectionery, bread, macaroni, foodstuff acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be the balneotherapeutic composition, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive of vitamin mixture, liquid for contact lenses, eye drops, basis of medical preparation, influencing different kinds of metabolism, including carbohydrate metabolism, phosphoric and calcium metabolism, homeostasis, hemopoiesis, hemostasis; agent, influencing immunity, correcting antitumoural therapy, antibiotics therapy, radio therapy used in gynecology, otorhynolaryngology, dentistry, ophthalmology, proctology, urology, for external use, dermatology; agent with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be the composition for cosmetic purpose, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be the composition for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be the composition for veterinary purposes, including feed and drink and/or medical preparation with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

The invented method could be used for stabilization of redox properties of water solutions and/or raw material-containing water, which could be used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.

In accordance with the third aspect the invention stipulates the use of amino-acids with not charged polar substitutes and/or their derivatives and/or peptides, containing mentioned amino-acids and/or their derivatives and/or their mixtures for stabilization of redox properties of composition, which corresponds to water solution and/or raw material-containing water with spontaneously changing redox properties, which are characterized by spontaneous increasing of the redox potential in relation to the potential of the hydrogen electrode, which value is considered as zero, in which mentioned amino-acids could be presented by glycine, serine, threonine, cysteine, tyrosine, asparagine or glutamine, and mentioned peptide could be presented by gelatin. Volume of amino-acids with not charged polar substitutes and/or their derivatives and/or peptides containing mentioned amino-acids and/or their derivatives and/or their mixtures could be more than 0.005% of weight.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be presented by foodstuffs, including mineral and/or drinking water, milk composition, juice, alcoholic and/or alcohol-free drink, mayonnaise, ketchup, sauce, meat, fish, vegetable and/or fruit semi-manufactured product, sausage or canned composition, confectionery, bread, macaroni, foodstuff acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be the composition for balneotherapeutic purposes, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive of vitamin mixture, liquid for contact lenses, eye drops, basis of medical preparation, influencing different kinds of metabolism, including carbohydrate metabolism, phosphoric and calcium metabolism, homeostasis, hemopoiesis, hemostasis; agent, influencing immunity, correcting antitumoural therapy, antibiotics therapy, radio therapy used in gynecology, otorhynolaryngology, dentistry, ophthalmology, proctology, urology, for external use, dermatology; agent with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be the composition for cosmetic purposes, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be compositions for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be compositions for veterinary purposes, including medical preparation, feed and drink and/or medical preparation with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

Amino-acids with not charged polar substitutes and/or their derivatives and/or peptides, containing mentioned amino-acids and/or their derivatives and/or their mixtures could be used for stabilization of redox properties of water solutions and/or raw material-containing water, which could be used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.

### DETAILED DESCRIPTION OF THE INVENTION

The purpose of this invention is the stabilization of redox properties of the composition corresponding to water, water solution and raw material-containing water with spontaneously changing redox properties, which are characterized by spontaneous increasing of the redox potential in relation to the potential of the hydrogen electrode, which value is considered as zero, for preservation of useful properties of mentioned composition.

There are known amino-acids with not charged polar substitutes in the structure of amino-acids, in which number there are glycine, serine, threonine, cysteine, tyrosine, asparagine, glutamine (see, for example, T. T. Berezov, B. F. Korovkin “Biological chemistry” Moscow, “Meditsina”, 1990, pp. 29-31).

Author of this invention all at once has found out that mentioned problem could be solved by addition in the composition corresponding to water, water solution and raw material-containing water with spontaneously changing redox properties, which are characterized by spontaneous increasing of the redox potential in relation to the potential of the hydrogen electrode, which value is considered as zero, of amino-acids with not charged polar substitutes in the structure of amino-acids, in which number there are glycine, serine, threonine, cysteine, tyrosine, asparagine, glutamine and/or their derivatives and/or peptides, containing mentioned amino-acids and/or their derivatives and/or their mixtures.

It should be noticed that amino-acids, mentioned in this invention, are the elements of proteins of the human and animals' body, plants and other organisms, they are not toxic and allowed for use as an article of food, component of therapeutic parenteral feed and other therapeutic and prophylactic agents, and they are used as nutritive additives (see, for example, A. P. Nechaev “Nutritive chemistry”, Sanct-Peterburg, GIORD, 2000, pp. 26-37, 371-373, 409-410).

In case of addition of mentioned amino-acids and/or their derivatives and/or peptides, containing mentioned amino-acids and/or their derivatives and/or their mixtures in composition corresponding to water, water solution and raw material-containing water with spontaneously changing redox properties, which are characterized by spontaneous increasing of the redox potential in relation to the potential of the hydrogen electrode, which value is considered as zero, it takes place the stabilization of specified redox properties, which is characterized by stabilization of the specified redox potential in relation to the potential of the hydrogen electrode, and this allows to keep useful properties of the mentioned composition for the period of not less than one year in dark place without special temperature conditions and to sterilize them in autoclave at temperature more then 120° C.

Stabilization of redox properties of the composition, corresponding to water, water solution and raw material-containing water according to this invention results in prevention of oxidation and microbiological deterioration of different surfaces after treatment of mentioned surfaces by the mentioned composition, and also provides protection of man, animals and plants from viruses, bacteria, fungi, moulds and peroxide oxidation of lipids in case of use of this composition by man and animals and treatment of plants by this composition, and also provides keeping of cells, including human and animals' semen, truncal cells, tissues and organs, intended, for example, for artificial insemination and transplantation, in viable conditions.

Applicants confirm useful properties of water solutions and raw material-containing water with redox properties, specified in this invention, experimentally by biological testing, as well as by instrumental methods.

The composition corresponding to this invention could be used in different fields, for example, in food industry, medicine, balneology, cosmetics, pharmaceutics, veterinary, animals breeding, fish farming, agriculture etc.

The composition with stabilized redox properties, produced on the basis of water solution and raw material-containing water with specified redox properties has the complex of unique biological and chemically active properties, which show themselves in:
Bactericide, bacteriostatic, antimycotic, antimould, virulicide, antiviral, antioxidant, antiphlogistic, antimutagenous, radio-protecting, immunostimulating, adoptogenous activity and in dissolving, regeneration and activating “friendly” micro flora of human and animals' organism, in particular, biphydobacteria and lactobacilli;
Keeping cells, such as truncal cells, and tissues and organs, intended, for example, for storing and transportation, for example, for further transplantation, in viable conditions;
In other useful properties and in keeping them not less than one year (in hermetically sealed containers).

In context of this invention the composition is the any known now or produced in future water solution and raw material-containing water with stabilized redox properties, which is characterized by stabilized redox potential in relation to the potential of the hydrogen electrode, with addition of specified amino-acids or their derivatives or peptides, containing one or more of mentioned amino-acids and/or their derivatives, or their mixtures in any combination.

Identification of the composition according to this invention includes the measurement of the redox potential of the composition, for example, by pH-meter or ion-meter, such as “pH-340”, “EW-74” etc., in presence of the negative redox potential of the composition and/or in presence of changing of both negative and positive redox potential in relation to the potential of hydrogen electrode, which value is considered as zero, the composition shall be analyzed on content of specified amino-acids, specified in this invention, for example, by use of chromatographic or spectrophotometric or other analysis for detection of amino-acids, specified in this invention, in the mentioned composition, of their derivatives or peptides, containing specified amino-acids and/or their derivatives, or their mixtures.

It is known that unbalanced redox properties of water solutions appear in case of deviation of the redox potential after electrochemical reduction of water solutions and raw material-containing water in comparison with the initial value of the redox potential by 50 mV, SCE, whatever initial redox potential the mentioned water solution and raw material-containing water was characterized by (see, for example, patent RF N2155717 of 01.28.2000).

According to this invention mentioned redox properties of the composition, which includes specified amino-acids, their derivatives and peptides, containing specified amino-acids and/or their derivatives, or their mixtures, are kept and appeared in case of deviation of the value of the redox potential of the composition by 50 mV, SCE, from condition of equilibrium, existing for water, water solutions and water containing materials, which were not subject to electrochemical (cathode) reduction, or in which there was non dissolved the biologically active additive Microhydrine etc.

If after mechanical impacts, for example, joggling or acceleration, after bubbling of gases through the composition, corresponding to this invention, including gases, which are not the oxidants, after electromagnetic and other impacts on the mentioned composition, or in case of its exposition, the redox potential of the mentioned composition is spontaneously increased by 50 mV, SCE, in relation to the potential of the hydrogen electrode, the value of which is considered as zero, the composition has all significant features of the mentioned invention and is the subject of the invention.

The composition in context of this invention could be also any composition of animals', vegetable, artificial and synthetic origin, known now or to be produced in future, containing water, water solution and raw material-containing water with stabilized redox properties, which are characterized by stabilized redox potential, which has mainly the negative value in relation to the potential of the hydrogen electrode, the value of which is considered as zero, in which there added the specified amino-acids or their derivatives or peptides, containing one or more on above mentioned amino-acids or their derivatives, and/or their mixture.

It could be:
Food raw materials for further production of fruit and vegetable concentrates, meat, fish, vegetable and fruit semi-manufactured products etc.;
Finished food products, for example, table and therapeutic mineral waters, drinking water, juices, alcohol-free and alcoholic drinks, milk compositions, mayonnaise, ketchup, sauce, meat, fish, vegetable and fruit compositions, confectionery, bread, macaroni, different canned compositions etc.
Medical raw materials for further production of medical preparations, in particular, different substances of animals', vegetable, artificial and synthetic origin, for example, the tissue of animal's pancreas for insulin production; root of liquorice for glyciram production; quinacrine for production of preparations for treatment of malaria, lupus erythematisis, skin leishmaniasis;
Finished medical preparations known now, for example, solutions for dialysis, nutritive mixtures, physiological salt solutions, artificial blood substitutes, liquids for keeping the contact lenses, any medical agents for external and internal use, for example, means influencing different kinds of metabolism, for example, carbohydrate metabolism, phosphoric and calcium metabolism, homeostasis, hemopoiesis, hemostasis and other kinds of metabolism; agents, influencing immunity, correcting antitumoural therapy, antibiotics therapy, radio therapy; agents using in gynecology, otorhynolaryngology, dentistry, ophthalmology, proctology, urology; agents for external use, for example, in dermatology; agents with disinfectant and/or antiseptic effect, preparation for treatment of disbacteriosis, antiphlogistic agent, antimicrobe agents for different groups, antiviral agents, antituberculous agents, antimycotic agents, agent used in gastroenterology and in hepatology, bronchopulmonary agents, antiallergic agents etc., and also physiologic salt solutions, parenteral agents for rehydration and detoxication, agents for correction of electrolytic and acid-alkaline balance, agents for parenteral alimentation, multivitamin agents with complex of biogenic adoptogens, amino-acid preparations using in case of functional asthenia, corrective food additives, plasma substituting and other artificial blood substitutes, as well as other medical agents, eye- and ear-drops, different aerosols, creams, ointments and gels;
Cosmetic raw materials for further manufacturing cosmetic products, including on the basis of liposomes and microcapsules, perfluo-hydrocarbons;
Cosmetic products, for example, tooth pastes, creams, including on the basis of liposomes and microcapsules, perfluo-hydrocarbons, and also gels, aerosols, perfume, eau-de-Colognes, lotions, deodorants, wet hygienic napkins, napkins, shampoo, conditioners;
Balneological compositions, for example, different mineral waters, therapeutic mud, sludge, peat;
Foodstuff and feed for animals' breeding, home animals and fish farming, for example, semi-manufactured products for preparation of feed, finished compositions, including canned, prophylactic and therapeutic preparations for animals' breeding, home animals and fish farming, drinking water for animals and water for aquariums.
Fertilizers for seeds couch, for different plants, including decorative.

It is not the full list of use of composition according to this invention, there are other ways to use it as food, therapeutic, cosmetic and other kinds of raw materials and finished products.

Composition according to this invention is produced as a rule by dissolution of specified amino-acids or their derivatives or peptides, containing specified amino-acids, and/or derivatives of above mentioned amino-acids or their mixtures in any combination according to the invention in water, water solution and raw material-containing water with spontaneously changing redox properties, which are characterized by spontaneous increasing of redox potential in relation to the potential of the hydrogen electrode.

Method of production of the composition according to this invention consists in mixing of amino-acids with not charged polar substitutes in the structure of amino-acids, which include glycine, serine, threonine, cysteine, tyrosine, asparagine, glutamine and/or their derivatives and/or peptides, containing mentioned amino-acids and/or their derivatives and/or their mixtures in any combination with water, water solution and raw material-containing water with spontaneously changing redox properties, which are characterized by spontaneous increasing of redox potential in relation to the potential of the hydrogen electrode, the value of which is considered as zero. The specified method provides preservation of different biologically and chemically active properties of mentioned composition for the long period.

The method includes different optional operations, which could be useful in this invention, for example, operations of dilution and condensing. Operation of dilution could be actually executed both by manufacturer and by user. Dilution could result in changing of redox properties, which causes increasing of redox potential, which is the measure of redox properties of water solutions. This method does not require special limitations on dilution and condensing, subject that it does cause decreasing of preservation of mentioned redox properties of water solut6ion and raw material-containing water according to this invention.

Other optional operations, useful for this invention, include operations of addition an/or mixing of any optional appropriate component, such as components listed below in section “Optional ingredients”.

Optional Ingredients.

Optional ingredients according to this invention are:
All ingredients, which could be used facultatively and are added by manufacturer of the composition for improvement of appearance, smell, taste, odor, consistence, and substances, which are added to the composition for acceleration and facilitation of technological process, and also substances, which influence technology of preparation and/or action of cosmetic and medical products, based on specified composition.
Natural and synthetic dyes, for example, curcumine (turmeric), riboflavin, carmine; chlorophyll and copper complexes oa chlorophyll; sugar dyes 1, 2, 3, 4; carotenoids, including beta-carotine, lycopine etc.; extracts of annato; oil-tars of paprika; lutein; been red (betanin); anthocyanins; tartrazine; quinoline yellow; yellow “Sundown” FCF; camuazine (azorubin); ponso 4R (bright red 3K), patented blue V; indigo carmine (indigotin); bright blue FCF; bright black BN (brilliant black) and other;
Stabilizers (fixers) of dyes, for example, nitrates and nitrites, for example, nitrites (NaNO3 and/or KNO3) and/or nitrates (NaNO2 or KNO2);
Aromatizers: for example, natural ether oils and extracts (oleoresin), rose, geranium etc.;
Food aromatizers, for example, arovanillon (ethyl vanillin), para-oxy-phenyl-3-butanone, citral, benzaldehyde, ethyl-2 methyl butyrate, allyl disulfide, anethole etc.;
Amplifiers of taste and odor, for example, sodium glutamate, lyzine hydrochloride, leucine, maltol, sodium chloride, as well as proteolytic enzyme preparations, for example, lipase;
Acid-forming products, for example, fruit acids, in particular, citric, malic, acetic, succinic acids, as well as hydrochloric, sulfuric, phosphorous acids and their salts etc.;
Intensive sweeteners and sugar substitutes, for example, acesulfam (E 950), aspartam (E 951), saccharin and its sodium salt (E 954), ciclamic acid and its salts (E 952), isomaltite (E 953), xylite (E967), mannite (E 421), fructose, saccharose, honey, glucose, galactose;
Substances for control of consistence, for example, ethers of polyoxyethylenesorbitan (E 432 . . . E 436) ammonium salts of phosphatidilic acid (E 442), mono- and diglycerides of fatty acids (E 471), phospholipins, ethers of glycerin and acetic and fatty acids (E 472), ethers of saccharose and fatty acids (E 473), other ethers of glycerin (E 474 . . . E 477), sodium lactylate (E 481(1)), calcium lactylate (E 482), ethers of sorbitan, SPAN's (E 491 . . . E 496);
Stiffeners and gel-forming agents, for example, acid polysaccharides with remains of uronic acid, polysaccharides with remains of sulfuric acid, neutral polysaccharides, acid hydrochlorides with remains of uronic acid (for example, tragacant E 413 and gum arabic E 414), as well as neutral compositions (for, example, resin of legumes of locust E 410 and guar E 412, as well as agar, carraginan, alginic acid and its salts, as well as modifies starches (E 1400 . . . 1405, E 1410 . . . 1414, E 1420 . . . 1423, E 1440, E 1442, E 1443, E 1450), complex ethers of cellulose E 461 . . . 467, including CMC (carboxynethyl cellulose), polysaccharides of microbe origin, for example, xanthane E 415, hellan resin E 418, alginic acid E400 and its salts E 401 . . . 404, pectin E 440, hialuronic acid and its salts;
Moisture keeping agents, for example, glycerin, sorbite, invert sugar and sugar-like substances, agar, alginates, pectins;
Film-forming stiffeners and gel-forming agents, as well as dispersions of polymers, glycerin, mono- and diglycerids of fatty acids, natural and synthetic waxes, lanolin, paraffin;
Regulators of acidity, for example, acids, alkalis, buffer salts;
Emulsifying salts, for example, phosphates, in particular, polyphosphates, citrates, tartrates, lactates;
Loosening agents, for example, yeast, ammonium, sodium bicarbonate;
Agents for filtering, for example, absorbents, flocculants (including cellulose, kieselguhr, pearlite);
Clarifiers, for example, agar, activated coal, corraginan, cellulose, gelatin, fish glue, charcoal, dried white of egg, kaolin, potassium ferrocyanide, kieselguhr, phytic acid, polyvinyl pyrrolidone, tannin, sodium pectate, furcelleran;
Capsulating agents, for example, gelatin, casein, gum arabic, pectin, carboxymethyl cellulose, fats and polymers, as well as mixtures of emulsifiers and hydrocolloids, and glycerin, sorbite, resins and sacchars as plasticizers;
Tablet agents, for example, fillers, separators, moisture keeping agents, absorbents, accelerators and inhibitors of dissolution, stabilizers, dyes and taste and odor agents;
a) Fillers—starch, amylose, micro-crystal cellulose, dicalcium phosphate, lactose, magnesium oxide, mannitol, polyglycols, sacchars and sugar substitutes, sorbite, manntol, grape sugar or water dissoluble ethylene glycol;
b) Separators (lubricants)—PAV, powdered cellulose, paraffin, catyl alcohol, stearic acid, stearates, talcum, polyethylene glycol;
c) Accelerators of dissolution—modifies starches, powdered cellulose, micro-crystal cellulose, methyl and ethyl cellulose, croscaramellose, alginic acid, potassium alginate insoluble, pectin, tragacanth, agar, sodium alginate;
d) Absorbents—starches, lactose, cellulose, kaolin, bentonit, high dispersed pyrogenic silicic acid;
e) Inhibitors of dissolution—solid paraffin, stearin, cacao oil, carboxymathyle cellulose in large amount, polyethylene glycol and polyvinyl pyrrolidone;
Dispersers, for example, solubizators and instatizators;
Antioxidants and protective gases, for example, sorbic acid and its salts, bezsoic acid and sodium benzoate, methyl, ethyl, propyl ethers of n-oxybenzoic acid; formic acid, sulfurous anhydride and sodium and potassium sulfites; sodium o-phenylphenol and o-phenylphenolat; diphenyl; carbon dioxide; nitrogen; vitamins E, C, A, H, B-group; butyl (hydro) oxyanisole (BOA, E 320); butyl (hydro)oxytoluene (BOT, “ionol” E 321), as well as isoascorbic (erithorbic) acid (E 315), sodium isoascorbat (E 316), tretbutyl hydroquinone (E 319) and ethers of gallic acid (E 310 . . . 313); antibiotics, for example, nisine; polyphenolic compounds, for example, epicatechin, epigallocatechin, epigallocatchin-gallate, bioflavonoids-ellagic, anthraflavonic, gallic acids, as well as epigenine, myricetin,; gllucosinolats, for example, isothiocyocyanates, indol-3-carbinol, sinigrin, brassinin and also sulfites, for example, diallyl sulfite, allildisulfite, allylmethyldisulfite, allylmethyltrisulfite; and also monoterpene compounds—d-limonene, aurapten, carveol, uroterpenol, serberol.

### Addition of Optional Ingredients

Preferably composition according to this invention corresponds to water and raw material-containing water with stabilized redox properties, which are characterized by stabilized redox potential mainly with negative value in relation to the potential of hydrogen electrode, which value id considered as zero, the method of production of which allows presence of at least one additional optional operation of mixing and/or operation condensing and/or operation of dilution, during which there is added the ingredient selected from the group, which consists of dyes, stabilizers (fixers) of dye, whiteners, aromatizers, amplifiers of taste and odor, table salt, sugar, proteins, acids, alkalis, buffers, salt substitutes, sugar substitutes (sweeteners), emulsifiers, stiffeners and gel-forming agents, preservation agents, antioxidants, thickeners, moisture keeping agents, anti-packing agents, film forming agents, acid regulators, foam suppressing agents, emulsifying salts, loosening agents, agents for filtering, clarifiers, extragents, carriers, diluters, solvents, separators, dryers, cooling and freezing agents, substances for vital activity of useful microorganisms, propellants, enzymes and enzyme preparations, catalysts of hydrolysis and inversion, dispersers, and from other groups with optional ingredients, presence of which in composition corresponding to this invention is not obligatory, but which provide to the composition some commercial value.

The specified composition for treatment of raw material could be produced by above-mentioned methods, which does not limit its production by these examples.

For the purpose of stabilization of redox potential of water solutions and/or raw material-containing water with spontaneously changing redox properties, which are characterized by spontaneous increasing of the redox potential in relation to the potential of the hydrogen electrode, which value is considered as zero, the best realization of the invention is the use of the amino-acid glycine in concentration of 0.1-0.5% of weight.

The invention is illustrated by the following examples, which does not limit it. Examples described below are the preferable; they are intended to confirm the possibility of realization of the invention and shall not by the basis for limitation of volume of assertions of the Applicant. Specialist in this brunch of industry will easily find out the possibilities of other realizations of the invention, which unconditionally are included in scope of assertions of the Applicant, reflected in the formula of the invention, described below.

### EXAMPLE 1

This example confirms the well-known thesis, according to which a water solution, which has the spontaneously changing redox properties, which are characterized by spontaneous increasing of redox potential in relation to the potential of the hydrogen electrode, the value of which is assumed as zero, will keep these properties during 1-20 days. Water from water supply system with initial characteristics: Eh=+260 mV, SCE, and pH=6.7, is directed to the device of “Izumrud”-type, which is manufactured in Russia. On the output characteristics of water have the values: Eh=−150 mV, SCE, pH=7,8-7,9. Mineralization of water is 170-190 mg/litre. Obtained water is poured into a non-transparent flask, sealed hermetically, and placed into refrigerator at temperature t=+4° C. out of light. In refrigerator the flask is not opened, not agitated and not influenced by other impacts. 72 hours later the flask is opened and the redox potential is measured. Its value is: Eh =+260 mV, SCE, and pH=6.8. Such experiments were conducted many times with the similar results. Other experimenters obtained the same results.

### EXAMPLE 2

This experiment proves stabilization of redox properties of water solution and raw material-containing water by amino-acids with not charged polar substitutes in the structure of amino-acids, in which number there are glycine, serine, threonine, cysteine, tyrosine, asparagine, glutamine or their derivatives, such as glycine amide, or peptides, such as, for example, gelatin. Water from water supply system with initial characteristics: Eh=+260 mV, SCE, and pH 6.7, is directed to the device of “Izumrud”-type, which is manufactured in Russia. On the output characteristics of water have the values: Eh=−150 mV, SCE, pH=7,8-7,9. Mineralization of water is 170-190 mg/litre. Obtained water with spontaneously changing redox properties is poured into 27 flasks, 3 flasks in each group (totally 9 groups), and amino-acids, corresponding to this invention, in particular, glycine, serine, threonine, cysteine, tyrosine, asparagine, glutamine and their derivatives, in particular, glycine amide, and peptides, in particular, gelatin, are added in different concentrations in each group of flasks, in particular, 0.005% of weight, 0.5% of weight, 10% of weight, and dissolved. Flasks are sealed hermetically and sterilized in autoclave at temperature more than 10⁵° C. during 18 hours. After sterilization these flasks with mentioned water solutions are put into the thermostat at temperature 50° C. Flasks with water solutions corresponding to this invention and stored at the mentioned temperature, protected from light, during 6 months. After 6 months flasks are opened, and the redox potential is measured.

TABLE 1
Stabilization of redox properties of water solution

| | Concentration of amino-acids, their | | | | | |
|---|---|---|---|---|---|---|
| | derivatives and peptides in water solution | | | | | |
| Name of | 0.005% | | 0.5% | | 10% | |
| amino-acid, | of weight | | of weight | | of weight | |
| Their | Eh | pH | Eh | pH | Eh | pH |
| derivatives | after 6 | after 6 | after 6 | after 6 | after 6 | after 6 |
| and peptides | months | months | months | months | months | months |
| Glycine | −15 | 7.2 | −270 | 7.9 | −170 | 7.7 |
| Serine | −5 | 7.1 | −160 | 7.8 | −110 | 7.6 |
| Threonine | −20 | 7.1 | −120 | 7.5 | −20 | 7.4 |
| Cysteine | −10 | 7.3 | −110 | 7.8 | −10 | 6.9 |
| Tyrosine | −5 | 7.2 | −80 | 7.4 | −30 | 7.2 |
| Asparagine | 0 | 5.9 | −60 | 6.9 | +20 | 7.0 |
| Glutamine | 0 | 5.8 | −50 | 7.2 | −10 | 7.2 |
| Glycine amide | +10 | 6.5 | −70 | 7.4 | +40 | 7.0 |
| Gelatin | +70 | 6.8 | −1 | 7.3 | −140 | 7.6 |

From practical point of view the use of gelatin is of interest as the composition corresponding to this invention, which in addition to the property of stabilization has the gel forming property. Complex of such properties allows to produce foodstuffs on the basis if gelatin with high viscosity due to binding of water with mentioned redox properties. Such compositions are jellies, sulzes, ice creams and different kinds of food.

### EXAMPLE 3

This Example Models Activity of Water Solutions Corresponding to this Invention in Organism for Demonstration of Stabilization of Redox Properties of Mentioned Solutions During their Dilution in Water Sector, which is Characterized by Positive Redox Potential.

Water from water supply system with initial characteristics: Eh=+250 mV, SCE, and pH=6.7, is directed to the system of water purification with the reverse osmosis, produced in USA, and has on output the following characteristics: Eh=+320, pH=6.3-6.7 and mineralization up to 1-6 mg/l. Obtained water is directed to the device of “Izumrud”-type, produced in Russia. On the output it has the following characteristics: Eh=−35 mV, SCE, pH=7.2-7.7 with mineralization up to 5 mg/litre. This water solution with spontaneously changing redox properties is poured into two 200 ml measuring glasses in volume of 100 ml in each glass. In one measuring glass (experiment) there is added the stabilizer, in particular, glycine, in concentration of 0.5% and dissolved. Into the other glass (control) the stabilizer is not added.

After this 100 ml of water, purified by the system of water purification with reverse osmosis with the redox potential Eh=+320 mV, SCE, are added into each measuring glass. Obtained solutions are sealed and are mixed.

10 minutes after mentioned operation of mixing redox potentials Eh of experimental and control solutions are measured. Results are in Table 2.

TABLE 2
Results of dilution

| Water solutions | Eh (initial) | Eh after dilution |
|---|---|---|
| Experiment | Eh = −35 mV | Eh = −10 mV |
| Control | Eh = −35 mV | Eh = +80 mV |

Results in Table 2 show that after dilution of experimental and control water solutions by water, purified by the system of water purification with the reverse osmosis, in proportion 50/50, the value of redox potential of the experimental solution remained in the area of negative potential, i.e. the solution still has expressed reduction properties, while the redox potential of control sample passed into area of positive values, i.e. water has the oxidative properties.

After measurement of Eh of control and experimental samples water solutions from mentioned measuring glasses were poured into open Petri dishes of standard dimensions and were exposed to the open air during two hours. After this the redox potential of experimental and control samples war measured again. Results of the experiment are in Table 3.

TABLE 3
Results of exposition to the air

| | Eh before | Eh after two hours |
|---|---|---|
| Water solutions | exposition to air | exposition to air |
| Control | +80 mV | +170 mV |
| Experiment | −10 mV | −03 mV |

Results in Table 3 show that the speed of increasing of redox potential, which is the measure of redox properties, in composition, corresponding to this invention, is Eh=(10−3) δ 2 hours=3.5 mV/hour, while for the control sample the speed of increasing of redox potential is Eh=(170-80): 2 hours=45 mV/hour.

### EXAMPLE 4

This Example Demonstrates Antioxidant Properties of Water Solution, Stabilized in Accordance with this Invention.

There were two samples of water, one of which was the control sample under No 2. The sample No 2, which had the initial redox potential Eh=−200 mV, SCE, after sterilization during 60 minutes at t=+120° C. was kept in hermetic container during 2 months. After expiration of this time the redox, potential Eh and pH of the sample were measured. They were: Eh=+240 mV, pH=8.22.

The sample No 1 was prepared simultaneously with the sample No 2. The difference is in the fact that just after production the sample No 1 with Eh=−200 mV, SCE, was stabilized by amino-acid glycine in concentration of 0.5% of weight, and after stabilization was also sterilized during 60 minutes with the following storing in hermetic container during 2 months. After two months both samples were ultra-filtered for purification from microbe contamination. Using these samples of water there were prepared two samples of milk by adding and mixing the milk powder—No 1 and 2 correspondingly. Both milk samples were left standing during fifteen days at room temperature in dark place. After that fats from both samples were extracted by diethyl ether. Fats' extracts were dried by sodium sulfate; ether was removed at temperature 30° C. Extract was placed in form of film between two glasses of potassium bromide. And infrared (IR) spectra were scanned within the range of 4000-400 cm⁻¹. By the same way there were registered spectra of fats extracted from: a) fresh cow butter; b) oxidized layer on the surface of butter, which was left standing during 15 days at room temperature. IR spectrum of fats' extract from milk (sample No 1) is similar to IR spectra of fats' extract from non-oxidized cow milk. Spectrum of fats' extract from milk (sample No 2) has the following difference: absorption band 1170 cm⁻¹ (C—O oscillations) has changed. The same changes of IR spectra were registered in the spectra of fats' extract from oxidized cow milk. Obtained data show that in the milk prepared with use of specially prepared water (sample No 1) oxidation of fats in composition of the milk is not observed.

Obtained data confirm that the composition corresponding to this invention, which includes raw material-containing water in form of milk, which consists of complex of inorganic and organic substances, with spontaneously changing redox properties, which are characterized by spontaneously increasing of the redox potential in relation to the potential of the hydrogen electrode, the value of which is considered as zero, has the antioxidant properties.

### EXAMPLE 5

This Example Demonstrates the Antioxidant Properties of Water Solution, Stabilized in Accordance with this Invention.

Two samples of cathode reduced water were prepared in the device “Izumrud” of Russian production and purified from microbe contamination by ultra-filtering. pH and redox potential (Eh) of samples were measured. Sample No 1: pH=7.0, Eh=−200 mV; sample No 2: pH=7.0, Eh=−200 mV. Sample No 1 was stabilized by amino-acid serine with concentration 0.1%. On the basis of these samples of water there were prepared two samples of milk by adding and mixing the milk powder: samples 1 and 2 correspondingly. Both milk samples were left standing during fifteen days at room temperature. After 15 days the sample of the milk No 1 was not oxidized, was not curdled and had all organoleptic properties of the fresh milk. The sample of the milk No 2 was completely oxidized and curdled.

Obtained results confirm that the composition corresponding to this invention, which includes raw material-containing water in form of milk, which consists of complex of inorganic and organic substances, with spontaneously changing redox properties, which are characterized by spontaneously increasing of the redox potential in relation to the potential of the hydrogen electrode, the value of which is considered as zero, has the antioxidant properties.

### EXAMPLE 6

This Example Demonstrates Bactericide Properties of Water, Water Solution and Raw Material-Containing Water, Stabilized in Accordance with this Invention.

3 (three) samples of electrochemically (cathode) reduced water were prepared from water supply system in the device “Izumrud” of Russian production. pH, redox potential (Eh) and mineralization were measured for all samples. Sample No 1: pH=7.0, Eh=−200 mV, mineralization 170-190 mg/l; sample No 2: pH=7.0, Eh=−200 mV, mineralization 170-190 mg/l; sample No 3: pH=7.0, Eh=−200 mV, mineralization 170-190 mg/l. All samples were sterilized during 60 minutes at temperature t=+120° C. The sample No 1 was stabilized by mixture of amino-acids, in particular, by glycine, serine, threonine, cysteine, tyrosine, asparagine, glutamine with total concentration 0.5%. The sample No 2 was not stabilized. The sample No 3 was stabilized by amino-acid glycine in concentration 0.5%. All samples were kept during 30 days at room temperature in dark place in hermetically sealed flasks.

On the basis of these samples there were prepared samples of milk by adding and mixing milk powder produced in New Zealand—No 1, 2, 3 correspondingly. The sample No 3 was placed in thermostat at temperature 37° C. for a long term (4 months) storage. One day later after samples of milk preparation there was made the microbe analysis of the sample No 1 and the sample No 2, which has shown that in the sample No 1 content of different bacteria is 10000 (ten thousand)-100000 (one hundred thousand) times lower, than in the sample No 2, in particular Streptococcus laticus, Staphylococcus sp., Micrococcus sp., Bacillus sp. etc. There was considerably decreased not only the amount of conditionally pathogenic microorganisms, but also microorganisms, which are characterized by hemolytic activity, in particular, B. Cereus, S. Aureus, E. Coli (enteropathogenic).

After execution of mentioned microbe analysis there were checked the values of redox potential Eh and pH of the sample No 1 and the sample No 2. They were for the sample No 1: Eh: =−200 mV, Ph=7.0, for the sample No 2: the redox potential and pH were correspondingly Eh=+250 mV, pH=7.9.

After 2 moths the sample No 3 was opened and Eh and pH were measured. They were correspondingly: −120 mV and 4.7. Measurements of these values were carried out in conditions favorable for contamination of milk by microorganisms. After mentioned measurements the sample was closed once more, but without strict sealing and put in thermostat at temperature t=+37° C. for 2 months.

4 months after beginning of the experiment there was conducted analysis of the sample No 3 in respect to microorganisms. It has shown that this sample contains only useful lactobacilli in concentration of 10⁵, in spite of conditions favorable for development of microorganisms (t=+37° C., good nutritive media, which includes milk fat, lactose, inorganic substances etc.).

So milk compositions, prepared on the basis of electrochemically (cathode) reduced water solution and stabilized in accordance with this invention, in absence of pasteurization and sterilization, which unfavorably influence the quality of milk compositions, are free of dissemination by pathogenic micro flora even in conditions of long term storing.

### EXAMPLE 7

This Example Demonstrates the Antimicrobe and Fungicide Activity of Water, Water Solution and Raw Material-Containing Water with Redox Properties, Specified in this Invention, and Also Demonstrates that Water, Water Solution and Raw Material-Containing Water, Stabilized in Accordance with this Invention, Along with Antimicrobe and Fungicide Activity, Activate the Growth of “Friendly” Micro Flora—Biphydobacteria and Lactobacilli.

There were used: water from water supply system with initial characteristics: Eh=+260 mV, SCE, device of reverse osmosis, which decreases mineralization of water from water supply system, and device “Izumrud” of Russian production. Water from water supply system is directed into the device “Izumrud” of Russian production and has on output the values from Eh=−50 mV to Eh=−250 mV, SCE, and pH=5.0 to pH=8.8 with mineralization 80-240 mg/l. Obtained water solutions with spontaneously changing redox properties are stabilized by amino-acid glycine with concentration of 0.1-0.5% and sterilized during 2 hours at temperature t=120° C. After this the mentioned water solutions were kept in hermetically sealed flasks during 120 days at t=24° C. in dark place. Different samples of mentioned reduced water solutions, stabilized according to this invention, were investigated in respect to antimicrobe and fungicide activity and influence on biphydobacteria and lactobacilli a day after exposition of mentioned microorganisms in mentioned water solution.

As test-microbes there were selected the following:
Lactobacilli, biphydobacteria—obligatory representatives of the normal enteric micro flora (Lactobacillus fermentum, Bifidobacterium sp,);
Colibacilli—from the content of the normal micro flora and conditionally pathogenic (E. coli O83, E. coli hemolytic);
Pathogens of enteric infections—salmonella, shigella, yersinia (Salmonella enteritidis, Shigella flexneri, Yersinia enterocolitica);
Conditionally pathogenic microorganisms—staphylococcus aureus, proteus, clebsiella, blue pus bacillus, listeria, microscopic fungi Candida, aspergilla (S. Aurtus, Proteus mirabilis, Klebsiella pneumoniae, Ps. Aeruginisa, Listeria monocytogenus, Candida albicans, Aspergillus niger);
And bacilli, clostridium and yeast (Bacillum subtilis, Clostridium sporogenes, Saccharamyces cerevisiae).

For cultivation in composition, stabilized according to this invention, there were prepared 18-24-hours suspension of microorganisms in three concentrations—10⁴, 10⁶, 10⁸ CFU/ml (CFU is the Colony Forming Unit of a microorganism per unit of volume). Microorganisms were washed off from the surface of nutritive medium by the buffer solution, which consists of 1000 ml of the distillated water, 0.45 g of the potassium dihydrophosphate and 5.34 g of the disodium hydrophosphate.

As a control in all experiments in parallel with the composition, corresponding to this invention, there was observed the amount of bacteria in the initial solution (10⁸ CFU) after 18-24 hours of thermostatic control.

There were obtained the following results in control solutions:
Colibacilli, yersinia, salmonella, clebsiella, blue pus bacillus, proteus—CFU was increased up to 10⁹;
Lactobacilli, biphydobacteria—CFU was decreased down to 10⁵-10⁴;
Amount of other microorganisms (staphylococcus, shigella, Candida, aspergilla, listeria, bacilli, yeast) CFU was on the prior level 10⁸.

Appropriate amount of microorganisms was put into test tubes with composition, corresponding to this invention, with hermetic plugs. Time of thermostatic control at 37° C. was 18-24 hours.

For quantitative control of bactericide (bacteriostatic) effect of the mentioned composition on microorganisms after thermostatic control, 0.1 ml of specifies solution was inoculated in three Petri dishes with the appropriate nutrient medium, and microorganisms were grown during 1 day (2-3 days for anaerobes). We used nutrient media of Russian production (Obolensk), of the firm BioMERIEUX (France), HiMedia (India), Serva (Germany): Endo, SS—for colibacilli, proteus, salmonella, shigella, yersinia, clebsiella; MRS—for lactobacilli; PALCAM—for listeria; Staph agar—for staphylococcus aureus; BIGGY—for microscopic Candida fungi; Pseudomonas agar—for blue pus bacillus; Thioglycolic medium—for biphydobacteria; Clostridial agar—for clostridium; 5% blood agar—for investigation of hemolytic properties of microorganisms and cultivation of aspergilla and bacilli; Czapek agar—for yeast.

Results of investigations are shown in tables 4-12

Legend: pH—hydrogen index, M—mineralization, Eh—redox potential.

TABLE 4
Cultivation of bacteria in composition,
corresponding to this invention during 24 hours

| | Composition with stabilized redox properties | | | |
|---|---|---|---|---|
| | | | | Initial concentration/ |
| Composition | | M | Eh | Concentration after cultivation |
| No | pH | mg/l | mV | Name of microorganism |
| | | | | Candida albicans |
| No 3 | 8.84 | 240 | −230 | 10⁸-10¹; 10⁶-10¹; 10⁴-0 |
| | | | | E. coli |
| No 3 | | | | 10⁸-10⁶; 10⁶-10⁴; 10⁴-10² |
| | | | | Staphylococcus aureus |
| No 3 | | | | 10⁸—10^{8;} 10⁶—10⁶; 10⁴—10⁴ |
| | | | | Bifidobacterium |
| No 5 | 6.0 | 80 | −290 | 10⁸-10¹⁰; 10⁶—10⁶; 10⁴—10⁴ |
| No 4 | 6.25 | 78 | −50 | 10⁸-10⁹; 10⁶-10⁷; 10⁴-10⁵ |
| | | | | Lactobacillus |
| No 5 | | | | 10⁸—10⁸; 10⁶-10⁷; 10⁴-10⁵ |
| No 4 | | | | 10⁸-10⁶; 10⁶-10⁴; 10⁴-10³ |
| | | | | E. coli |
| No 5 | | | | 10⁸-10⁷; 10⁶-10⁵; 10⁴-10² |
| No 4 | | | | 10⁸—10⁸; 10⁶—10⁶; 10⁴-10³ |
| | | | | Pseudomonas aeruginisae |
| No 5 | | | | 10⁷-10⁶; 10⁵-10⁴; 10³-10¹ |
| No 4 | | | | 10⁷-10⁶; 10⁵-10⁴; 10³-10¹ |
| | | | | Proteus vulgaris |
| No 5 | | | | 10⁸-10⁵; 10⁶-10³; 10⁴-10¹ |
| No 4 | | | | 10⁸—10⁸; 10⁶—10⁶; 0⁴—10⁴ |

TABLE 5
Cultivation of bacteria in composition
corresponding to this invention during 24 hours

| | Composition with stabilized redox properties | | | |
|---|---|---|---|---|
| | | | | Initial concentration/ |
| Composition | | M | Eh | Concentration after cultivation |
| No | pH | mg/l | mV | Name of microorganism |
| | | | | Staphylococcus aureus |
| No 6 | 8.8 | 165 | −300 | 10⁶-10⁵; 10⁴-10²; 10²-10¹ |
| No 7 | 6.05 | 95 | −150 | 10⁶—10⁶; 10⁴—10⁴; 10²-10¹ |
| No 8, 9 | 5.90 | 124 | −150 | 10⁶—10⁶; 10⁴—10⁴; 10²-10¹ |
| No 8, 9(a) | 5.47 | 135 | −140 | 10⁶—10⁶; 10⁴—10⁴; 10²-10¹ |
| | | | | Candida albicans |
| No 6 | | | | 10⁶-10¹; 10⁴-0; 10²-0 |
| No 8, 9 | | | | 10⁶-10¹; 10⁴-0; 10²-0 |
| No 8, 9(a) | | | | 10⁶-10¹; 10⁴-0; 10²-0 |
| | | | | Lactobacillus |
| No 8, 9 | | | | 10⁶—10⁶; 10⁴—10⁴; 10²—10² |
| No 8, 9(a) | | | | 10⁶—10⁶; 10⁴-10⁵; 10²-10³ |
| | | | | E. coli |
| No 8, 9 | | | | 10⁸-10⁷; 10⁶-10⁵; 10⁴-10² |
| No 8.9(a) | | | | 10⁸—10⁸; 10⁶—10⁶; 10⁴-10³ |
| | | | | Klebsiella sp. |
| No 8, 9 | | | | 10⁸—10⁸; 10⁶—10⁶; 10⁴-10³ |
| No 8, 9(a) | | | | 10⁸—10⁸; 10⁶—10⁶; 10⁴—10⁴ |
| | | | | Pseudomonas aeruginisae |
| No 8, 9 | | | | 10⁸—10⁸; 10⁶—10⁶; 10⁴—10⁴ |
| No 8, 9 (a) | | | | 10⁸—10⁸; 10⁶—10⁶; 10⁴-10³ |

TABLE 6
Cultivation of bacteria in composition, corresponding
to this invention, during 24 hours

| | Composition with stabilizer redox properties | | | |
|---|---|---|---|---|
| | | | | Initial concentration/ |
| Composition | | M | Eh | Concentration after cultivation |
| No | pH | mg/l | mV | Name of microorganism |
| | | | | Candida albicans |
| No 14 | 8.8 | 152 | −150 | 10⁸-10¹; 10⁶-10¹; 10⁴-0 |
| | | | | E. coli |
| | | | | 10⁸-10⁷; 10⁶-10³; 10⁴-10² |
| | | | | Staphylococcus aureus |
| | | | | 10⁸—10⁸; 10⁶-10⁴; 10⁴-10¹ |
| | | | | Sigella sonnae |
| | | | | 10⁸-10³; 10⁶-10¹; 10⁴-0 |
| | | | | Lactobacillus |
| | | | | 10⁸—10⁸; 10⁶-10⁷; 10⁴-10⁶ |
| | | | | Salmonella sp. |
| | | | | 10⁸-10⁵; 10⁶-10²; 10⁴-10¹ |
| | | | | Pseudomonas aeruginisae |
| | | | | 10⁷—10⁷; 10⁵-10⁴; 10³-10¹ |

TABLE 7
Cultivation of bacteria in composition, corresponding
to this invention, during 24 hours

| | Composition with stabilized redox properties | | | |
|---|---|---|---|---|
| | | | | Initial concentration/ |
| Composition | | M | Eh | Concentration after cultivation |
| No | pH | mg/l | mV | Name of microorganism |
| | | | | Candida albicans |
| No 16 | 7.3 | 170 | −150 | 10⁸-10⁵; 10⁶-10¹; 10⁴-0 |
| | | | | E. coli |
| | | | | [symbol] |
| | | | | 10⁸-10⁷; 10⁶-10³; 10⁴-10¹ |
| | | | | Staphylococcus aureus |
| | | | | 10⁸—10⁸; 10⁶-10⁵; 10⁴-10³ |
| | | | | Sigella sonnae |
| | | | | 10⁸-10⁶; 10⁶-10²; 10⁴-0 |
| | | | | Salmonella sp. |
| | | | | 10⁸-10⁷; 10⁶-10³; 10⁴-10¹ |
| | | | | Pseudomonas aeruginisae |
| | | | | 10⁷—10⁷; 10⁵-10⁴; 10³-10¹ |

TABLE 8
Cultivation of bacteria in composition, corresponding
to this invention, during 24 hours

| | Composition with stabilized redox properties | | | |
|---|---|---|---|---|
| | | | | Initial concentration/ |
| Composition | | M | Eh | Concentration after cultivation |
| No | pH | mg/l | mV | Name of microorganism |
| | | | | Candida albicans |
| No 20 | 6.85 | 220 | −150 | 10⁸-10⁵; 10⁶-10³; 10⁴-0 |
| | | | | E. coli |
| | | | | 10⁸-10⁶; 10⁶-10⁴; 10⁴-10¹ |
| | | | | Staphylococcus aureus |
| | | | | 10⁸—10⁸; 10⁶-10⁴; 10⁴-10² |
| | | | | Sigella sonnae |
| | | | | 10⁸-10⁵; 10⁶-10²; 10⁴-0 |
| | | | | Salmonella sp. |
| | | | | 10⁸-10⁶; 10⁶-10²; 10⁴-10¹ |

TABLE 9
Cultivation of bacteria in composition, corresponding
to this invention, during 24 hours

| | Composition with stabilized redox properties | | | |
|---|---|---|---|---|
| | | | | Initial concentration/ |
| Composition | | M | Eh | Concentration after cultivation |
| No | pH | mg/l | mV | Name of microorganism |
| | | | | Candida albicans |
| No 21 | 7.0 | 170 | −90 | 10⁸-10⁶; 10⁶-10³; 10⁴-0 |
| | | | | E. coli |
| | | | | 10⁸-10⁷; 10⁶-10⁴; 10⁴-10² |
| | | | | Staphylococcus aureus |
| | | | | 10⁸—10^{8;} 10⁶-10⁵; 10⁴-10³ |
| | | | | Sigella sonnae |
| | | | | 10⁸-10⁶; 10⁶-10²; 10⁴-0 |
| | | | | Salmonella sp. |
| | | | | 10⁸-10⁷; 10⁶-10³; 10⁴-10¹ |

TABLE 10
Cultivation in composition, corresponding
to this invention, during 24 hours

| | Composition with stabilized redox properties | | | |
|---|---|---|---|---|
| | | | | Initial concentration/ |
| Composition | | M | Eh | Concentration after cultivation |
| No | PH | mg/l | mV | Name of microorganism |
| | | | | Candida albicans |
| No 24 | 7.2-7.4 | 190-200 | −150 | 10⁸-10⁶; 10⁶-10⁴; 10⁴-10¹ |
| | | | | E. coli |
| | | | | 10⁸-10⁷; 10⁶-10⁴; 10⁴-10³ |
| | | | | Staphylococcus aureus |
| | | | | 10⁸—10⁸; 10⁶-10⁵; 10⁴-10³ |
| | | | | Sigella sonnae |
| | | | | 10⁸-10⁵; 10⁶-10²; 10⁴-0 |
| | | | | Salmonella sp. |
| | | | | 10⁸-10⁶; 10⁶-10³; 10⁴-10¹ |
| | | | | Klebsiella pneumoniae |
| | | | | 10⁸-10⁷; 10⁶-10³; 10⁴-10² |
| | | | | E. coli (hemolytic) |
| | | | | 10⁸-10⁷; 10⁶-10⁵; 10⁴⁻10² |

TABLE 11
Cultivation in composition, corresponding
to this invention, during 24 hours

| | Composition with stabilized redox properties | | | |
|---|---|---|---|---|
| | | | | Initial concentration/ |
| Composition | | M | Eh | Concentration after cultivation |
| No | PH | mg/l | mV | Name of microorganism |
| | | | | Candida albicans |
| No 25 | 8.4 | 400 | −250 | 10⁸-0; |
| | | | | E. coli (hemolytic) |
| | | | | 10⁸-10⁷; |
| | | | | S. aureus |
| | | | | 10⁸-10⁴; |

TABLE 12
Cultivation in composition, corresponding
to this invention, during 24 hours

| | | | Initial concentration/ |
|---|---|---|---|
| | | | Concentration |
| | | | after cultivation |
| Physiological salt solution | pH | ORP | Helicobacter pylori |
| Physiological solution NaCl - 0.85% | 7.6 | +300 | 10⁶-10⁵ |
| (control) | | | |
| Physiological solution NaCl - 0.85% | 8.4 | −300 | 10⁶-10³ |
| (stabilized according to this | | | |
| invention) | | | |

### EXAMPLE 8

The Example, Demonstrating Treatment of Disbacteriosis by the Composition with Stabilized Redox Properties.

Influence of peroral use of composition corresponding to this invention with parameters: pH=7.5, Eh=−150 mV, mineralization 170-190 mg/l on composition of enteric micro flora was investigated on limited number of volunteers. In case of its administration in amount of 0.6 litre during four weeks there was observed stable composition of micro flora: number of biphydobacteria was in average 10⁷-10⁹ CFU/g, lactobacilli-10⁶-10⁷, normal colibacilli reached 10⁶-10⁷, number of conditionally pathogenic enteric microorganisms did not exceed 10¹, staphylococcus aureus and its other hemolytic kinds, microscopic fungi, aspergilla, conditionally pathogenic non fermenting bacteria were absent. Characteristics of the immune system of examined patients were within the limits of the age norm. For one of patients before the use of mentioned composition there were observed the following changes of enteric micro flora: decrease of number of lactobacilli (10⁴), biphydobacteria (10⁵) and normal colibacilli (10⁴), and increase of spore microorganisms (10⁸) and fecal streptococci (10⁸), including hemolytic (10⁴). Number of conditionally pathogenic enterobacteria (clebsiella) reached 10⁵. After administration of mentioned composition during four weeks there were observed the increase of number of lactobacilli, biphydobacteria and normal colibacilli. Clebsiella and hemolytic streptococci were not found, and number of spore microorganisms was decreased down to 10³.

Besides, before administration of mentioned composition there were found out small amount of pseudomonadas and candidas, which were eliminated from the enteric tract of the patient.

Examination of enteric tract of the patient V.D., who used mentioned composition during one month, has shown the stable composition of micro flora, although before its administration he has expressed clinical symptoms of disbacteriosis (alternate of constipation and diarrhea, meteorism and pain in large intestine).

Patient V.P. before administration of mentioned composition had the low number of lactobacilli (10⁴) and colibacilli (10⁴), and there were observed conditionally pathogenic clebsiella (10⁵), staphylococcus aureus (10²) and clostridium (10⁸). After four weeks of administration of stabilized composition there was observed the following content of enteric micro flora: number of lactobacilli has increased up to 10⁸, and normal colibacilli up to 10⁷, number of conditionally pathogenic clebsiella has decreased by two orders, and clostridium by four orders.

### EXAMPLE 9

The Example, Demonstrating Increasing of Sensitivity of Bacteria to Antibiotics after Cultivation in Composition with Stabilized Redox Properties.

It was examined resistance of staphylococcus aureus, salmonella, shigella and clebsiella to some antibiotics: chloramphenicol, kanamycin, amoxycillin, nalidixic acid, amicacin, gentamycin and ciprofloxacin. It was used the method of preparations diffusion into nutrient agar. Above mentioned antibiotics, dissolved in composition, corresponding to this invention with pH=7.9, mineralization 350 and Eh=−230 mV, SCE, were used for treatment of bacteria. It was used sterilized water with Eh=250 mV, SCE, pH=7.9 and mineralization 350 mg/l for control.

There were obtained the following results:
Salmonella sp.: strain has changed from resistant to sensitive to gentamycin and nalidixic acid and from medium sensitive to sensitive to ampicillin and ciprofloxacin;
Shigella sp. strain has changed from resistant to sensitive to ampicillin and gentamycin and from medium sensitive to sensitive to ciprofloxacin, amicacin and kanamycin;
S. aureus: sensitivity to ampicillin has increased;
Klebsiella Pneumoniae: sensitivity to amicacin had increased.

Table 13 shows % of increase of diameters of zones of suppression of microorganisms' growth in experimental water solution in comparison with the control one.

TABLE 13
Rate of increase of sensitivity of bacteria (%)

| | Salmonella | Shigella | Clebsiella | Staphylococcus |
|---|---|---|---|---|
| Ampicillin | 26 | 44 | 7 | 22 |
| Amicacin | 18 | 38 | 34 | 4 |
| Nalidixic acid | 42 | 33 | 11 | 3 |
| Gentamycin | 47 | 40 | 6 | 11 |
| Kanamycin | 18 | 35 | 12 | 8 |
| Chloramphenicol | 6 | 17 | 5 | 3 |
| Ciprofloxacin | 28 | 36 | 17 | 10 |

This example shows the presence of effect of increasing of sensitivity of microorganisms to antibiotics, dissolved in composition, corresponding to this invention. So contact of microorganisms with antibiotics, dissolved in the mentioned composition, results in increasing effect of treatment or disinfection by antibiotics. Antibiotics, specified in this example, do not limit the joint use of the composition, corresponding to this invention, with other antibiotics, disinfectant and other medical preparations, which are used now or will be used in future.

### General Conclusions for Examples 6-9

Investigations of influence of the composition, corresponding to this invention, with mineralization and pH, which do not exceed physiologically acceptable values, with pH=5.0-8.8, mineralization on the level up to 350 mg/l, Eh=from −50 mV to −300 mV, SCE, on life activity of microorganisms of different families, have shown the following:
1. The composition, corresponding to this invention, has the expressed bactericide effect in regard to salmonella, shigella and microscopic fungi Candida;
2. The composition, corresponding to this invention, considerably stimulates growth of lactobacilli, biphydobacteria and brewer's and baker's both in alkali and in acid cultivation medium;
3. The composition, corresponding to this invention, has shown the expressed bacteriostatic effect in regard to blue pus bacilli, staphylococcus aureus, clebsiella, proteus, aspergilla, listeria, clostridium and bacilli. This bacteriostatic effect is observed both in alkali and in acid medium. This fact distinguishes the mentioned composition from known conservation agents, which are effective in acid media, for example, benzoic acid, sodium benzoate, sorbic acid, potassium sorbate etc.;
4. Physiologic salt solution, based on the mentioned composition, takes bacteriostatic effect, including alkaline media, in respect to Helicobacter pylori, which causes development of peptic ulcer;
5. Investigated bacteria increase their sensitivity to antibiotics dissolved in the composition, corresponding to this invention;
6. Use of the composition, corresponding to this invention, for dilution of powder milk and contaminated milk products prevents reproduction of conditionally pathogenic bacteria or leads to their disappearance, including long exposition;
7. It is observed 18-20% decrease of zones of hemolysis of S. Aureus in result of treatment of bacterial suspension by stabilized composition, which indirectly indicates decrease of virulence;
8. It is shown that the bacteriostatic effect of the composition, corresponding to this invention, appears after 1 hour of its impact on bacteria.

Experiments with microorganisms, cultivated in the composition, corresponding to this invention, show that after addition of bacterial suspension, yeast-like microscopic fungi, mold fungi and yeast in concentration from 10⁴ to 10⁸ CFU/g in the mentioned composition results in:
Increasing of amount of useful bacteria (biphydobacteria and lactobacilli) (10-100 times);
Increasing of growth of yeast (10-100 times);
Decreasing of amount of microscopic fungi Candida from 10⁸-10⁴ to 0. Considerable decreasing of reproduction of salmonella and shigella: 100-1000 times, if initial concentration is 10⁸, and down to 0, if initial concentration is 10⁴.

So conducted investigations show perspective possibilities for wide use of the composition with stabilized redox properties in accordance with this invention for sanation of micro flora of enteric tract without side effects, connected with inclusion of products of vital activity of microorganisms and chemical fillers in known protobiotics and eubiotics, including antibiotics and conservation agents.

The composition, corresponding to this invention, could be used in food industry for preparation and dissolution of different powder-like products for normalization of micro flora or prevention of foodstuff contamination of their disinfection.

### EXAMPLE 10

This Example Demonstrates Virulicide and Antiviral Activity of the Composition, Corresponding to this Invention, and Simultaneously Confirms that this Composition is not Toxic.

There was used the cytopathogenic strain of the hepatitis C virus (HCV), related to the genotype lb. The strain was obtained from the blood serum of patient infected by chronic viral hepatitis C and identified as hepatitis C virus. There were used infectious doses of HCV equal to 10.0 TCD 50/20 mkl.

There were used sells cultures of kidneys of green marmoset highly sensitive to cytopathogenic action of HCV, clone No 6 (Vero-6).

They were used in form of one-day cells monolayer, grown in 24-cells plastic plates. Cells cultures Vero-6 were grown on double medium “Igla” with 10% of calf embryo serum with addition of glutamine and antibiotics (100 units/ml).

For titration of residual infectability of virus it was used the same line of cells of pig embryo kidney (SPEV) also sensitive to HVC reproduction. They were used in form of one-day cells monolayer, grown in 96-cells plastic plates cells cultures. Cells cultures SPEV were grown on 199 media with addition of 10% of cattle serum and antibiotics.

There were also used sells cultures of pig testickles (PDP). Cultures of sells PDP were used in form of one day cells mono-layer, grown in 24- and 48-cells plastic plates on minimal medium “Igla” (firm HY Glone, USA) with addition of 10% of calf embryo serum, glutamine and antibiotics.

There were used the composition with stabilized redox properties with Eh=−300 mV, pH=7.5-8.0 and mineralization 170-190 mg/l. Before the beginning of experiment 2 g of dry medium “Igla” were dissolved in water, carefully rotating the flask in horizontal position, after complete dissolution the liquid medium was slowly filtered so that filtrate layered on test tube walls. Than 7% calf embryo serum and antibiotics were carefully added to the filtrate. This medium, containing the composition with stabilized redox properties, was both the supporting media and nutrient medium for infected and non-infected cultures of cells. As control experiment there were used the same cultures of cells and virus, grown on media without mentioned composition.

The following experiments were executed:
1. Investigation of cytotoxic properties of the composition with stabilized redox properties. For this purpose cultures of cells Vero-6, SPEV and PTP were grown and left standing in medium containing the mentioned composition for 4 days. Obtained results of vitality of cells were compared with the same for cultures of cells, grown in standard medium.
2. Investigation of virulicide properties of the composition with stabilized redox properties. The virus containing liquid and the nutrient medium containing the mentioned composition were mixed in proportion 1:9 correspondingly and left standing for 1 hour and 24 hours at 4° C. As a control it was used the virus containing liquid mixed in proportion 1:9 with usual nutrient medium without mentioned composition. Results were obtained after titration of residual HCV infectious activity in cultures of cells in experimental and control samples.
3. Investigation of antiviral activity of the composition with stabilized redox properties. Antiviral activity of the composition, corresponding to this invention, was investigated in PTP cells cultures according to data of a) vitality of infected cells, growing on usual medium and medium, containing the mention composition, b) concentration of infectious virus, produced by cells, growing on usual medium and on medium with the composition, corresponding to this invention.

The following results were obtained:
1. Investigation of cytotoxic properties of the composition with stabilized redox properties. Obtained data show that medium, containing the mentioned composition, has no toxic properties and does not influence vitality, proliferative activity of non-infected cultures of cells Vero-6, PTP and SPEV. Obtained data show that cultures of cells, grown on medium, containing the mentioned composition, are characterized by the grater adhesive ability of cells monolayer (ability to be fixed on the culture flask bottom), which could indicate the grater vitality of cells, grown in such conditions.

2. Investigation of virulicide properties of the composition with stabilized redox properties. These data are shown in table 14.

TABLE 14
Virulicide properties of the composition
with stabilized redox properties

| | Titers of HCV in the medium of cultures of | | |
|---|---|---|---|
| | cells with and without the composition | | |
| | with stabilized redox properties (lg TCD50/ml) | | |
| | | Medium with the composition | Lg of |
| | Control | with stabilized redox | decreasing of |
| Culture of cells | medium | properties | HCV titer |
| Vero-E6 (24 h) | 11.5 | 8.5 | 3.0 |
| SPEV (1 h) | 6.3 | 4.0 | 2.3 |
| PTP (24 h) | 2.8 | No1 - 0 | 2.8 |
| | | No2 - 0.5 | 2.3 |

24 hours—incubation of virus during 24 hours at t=+4° C.; 1 hour—during 1 hour in the same conditions; No 1 and No 2—samples of composition, corresponding to this invention.

As it is shown in table 14 exposure of material, containing HCV, in medium, containing the composition, corresponding to this invention, during 24 hours at +4° C. results in decreasing of infectious activity of virus for cultures of cells of different origin by 2.8-3.0 lg TCD50. Activity of mentioned composition in case of exposure during 1 hour is some lower (decreasing of HCV titers by 2.3 lg TDC50). So the data, presented in table 14, show that the medium “Igla” based on composition, corresponding to this invention, is characterized by virulicide activity.
3. Antiviral properties of the composition with stabilized redox properties in respect to infection, caused by HCV in cultures of cells PTP.

Experimental data on antiviral properties of the composition, corresponding to this invention, are shown in tables No 15, 16, 17.

TABLE 15
Vital activity of HCV-infected cultures of cells PTP, cultivated on
medium with the composition, corresponding to this invention.
Introduction of medium, containing the composition, corresponding
to this invention, just after infection of cells by HCV

| | % of survived | | |
|---|---|---|---|
| | HCV-infected | | |
| | cells in repeated | | |
| | experiments | | |
| | on 3^{rd} day | | |
| Variants of | after infection. | | |
| experiment | A | B | C |
| Experiment No 1 | 100 | 100 | 100 |
| Experiment No 2 | 100 | 75 | 100 |
| Experiment No 3 | 50 | 50 | 50 |
| Control of cells | 100 | 100 | 100 |

Experiment No 1 - the use of the composition, corresponding to this invention, under No 1
Experiment No 2 - the use of the composition, corresponding to this invention, under No 2
Experiment No 3 - the use of standard tridistillated water; A, B, C - repeated experiments.

It was found out that growing of HCV-infected cultures cells PTP on the medium, containing the composition, corresponding to this invention, which was added just after infection of cells, results, as rule, in 100% survival of cells. In control experiments at the same day 20% of HCV-infected cells in monolayer have died. These data evidence in favor of antiviral properties of the mentioned composition.

TABLE 16
Vital activity of HCV-infected cultures of cells PTP, cultivated on
medium, containing the composition with stabilized redox properties
Introduction of medium, containing the composition, corresponding
to this invention, 24 hours before infection of cells by HCV

| | % of survived | | |
|---|---|---|---|
| | HCV-infected | | |
| | cells in repeated | | |
| | experiments | | |
| | on 3^{rd} day | | |
| Variants of | after infection. | | |
| experiment | A | B | C |
| Experiment No 1 | 100 | 100 | 100 |
| Experiment No 2 | 100 | 100 | 100 |
| Experiment No 3 | 50 | 50 | 50 |
| Control of cells | 100 | 100 | 100 |

Experiment No 1 - the use of the composition, corresponding to this invention, under No 1
Experiment No 2 - the use of the composition, corresponding to this invention, under No 2
Experiment No 3 - the use of standard tridistillated water; A, B, C - repeated experiments.

Data in table 16 show that growing of HCV-infected cultures cells PTP on the medium, containing the composition, corresponding to this invention, which was added 24 hours before infection of cells, also results in 100% survival of cells. In control experiments at the same day 50% of HCV-infected cells in monolayer have died. These data evidence also in favor of antiviral properties of the composition with stabilized redox properties.

TABLE 17
Antiviral activity of the composition with stabilized redox properties
on the model of HCV infection in cultures of cells PTP

| | Titers of hepatitis C virus (lg TCD50/ml | | | | | | |
|---|---|---|---|---|---|---|---|
| | for cultures of cells SPEV) in | | | | | | |
| Time of addition | samples of medium from cultures of | | | | | | |
| of the medium, | cells PTP on 3^{rd} day after infection. | | | | | | |
| containing the | Experi- | | Experi- | | Experi- | | lg of |
| composition with | ment | | ment | | ment | | decreasing |
| stabilized redox | No 1 | | No 2 | | No 3 | | of |
| properties | A | B | A | B | A | B | HCV virus |
| At the moment of | 1.5 | 1.5 | 1.5 | 0.8 | 5.0 | 5.2 | 3.5-4.4 |
| infection | | | | | | | |
| 24 hours before | 4.3 | 4.3 | 4.3 | 3.3 | 7.0 | 6.9 | 2.7-3.7 |
| infection | | | | | | | |

Notes are the same as in tables 15 and 16.

3) For direct estimation of antiviral effect of the composition, corresponding to this invention, in cultures of cells PTP, infected by HCV, on the 3^{rd} day after infection the medium was sampled and titrated on cultures of cells PTP. Table 17 shows the results of titration of samples of the cultural medium.

It is shown that the composition with stabilized redox properties has antiviral properties. In particular, it was found out that the mentioned composition has the maximal antiviral activity in case of treatment of cells by the mentioned composition both at the moment of infection and 24 hours after infection of cultures of cells by HCV. In these cases titers of virus in cultures of cells, treated by the mentioned composition just after infection, were decreased by 3.5-4.4 lg TDC50. Treatment of cells by the mentioned composition 24 hours before infection of cells, as rule, also results in significant antiviral effect (decreasing of HCV titers by 2.7-3.7 lg TCD50).

4) Investigation of virulicide and antiviral activity of the composition, corresponding to this invention, from flasks, filled not “up to edge” by water, which has contact with air.

In these experiments, as a rule, it was used the composition, corresponding to this invention, remaining in flasks after previous experiments, after 24-hours exposition with air with keeping the sterile conditions (sterile closed flasks).

In particular, it was shown that, in case of “aeration” of the mentioned composition in flasks during 24 hours, it completely loses virulicide and antiviral properties in regard to hepatitis C virus.

So:
1. The composition with stabilized redox properties has no cytotoxic properties for cultures of Vero-6, SPEV and PTP cells during 4 and more days of cultivation.
2. The composition with stabilized redox properties has the virulicide activity in regard to hepatitis C virus after exposition of liquid, containing HCV, and medium “Igla”, prepared on the mentioned composition, during 1 hour and 24 hours, titers HCV for cultures of cells were decreased by 2.3-3.0 lg TCD50.
3. The composition with stabilized redox properties has antiviral properties and is able to decrease HCV production by infected cells in average by 3.0-4.0 lg in case of its adding just after adsorption of HCV on cells or 24 hours before infection of cells by hepatitis C virus;
4. Virulicide and antiviral properties of the composition with stabilized redox properties were completely lost in case of aeration of the mentioned composition during 24 hours.

### EXAMPLE 11

This Example Proves the Possibility of Stabilization of Redox Properties of Beer, which is Characterized by Positive, Spontaneously Changing Redox Potential in Relation to the Hydrogen Electrode, the Value of which is Considered as Zero.

At brewery there were conducted measurements of Eh of fresh, not filtered and not pasteurized light and dark beer of upper fermentation after completion of fermentation (i.e. 22 days after beer was brewed). Measurements have shown:
Light beer—Eh=+50 mV, SCE;
Dark beer—Eh=+30 mV, SCE.

Light and dark beer were poured into flasks and stabilized by amino-acid glycine in concentration of 0.5% of weight and hermetically sealed (experimental beer). Ordinary light and dark beer, also hermetically sealed in flasks, were used as control (control beer). After 15 days there were conducted measurements for light and dark beer. Measurements have shown:
Experimental light beer: Eh=+60 mV, SCE;
Experimental dark beer: Eh=+40 mV, SCE;
Control light beer: Eh=+100 mV, SCE;
Control dark beer: Eh=+90 mV, SCE.

### EXAMPLE 12

Example, Demonstrating Stabilization of Hydrosulphuric Water and Hydrosulphuric Mud with Use of Amino-Acids, Specified in this Invention.

There were used artificial hydrogen sulfide—H₂S, produced by interaction of ferrous sulfide with diluted solution of hydrochloric acid FeS+2HCl>FeCl₂+H₂S, Adler sludge and water from water supply system with mineralization of 0.17 g/l, Eh=+290−(+300) mV, SCE, pH=7.2.

There were produced 19 litres of water, saturated with hydrogen sulfide, with Eh=−170 mV, SCE and pH=6.6. Amount of H₂S per 100 ml of solution was 340-370 mg. From this amount 2 litres were used for dilution for 30-fold decreasing of concentration of H₂S. It was obtained solution with Eh=−20 mV, SCE, and pH=8.7. Part of solutions both with Eh=−170 mV, SCE, pH=6.6 and Eh=−20 mV, SCE, pH=8.7 was used for creation of control group of samples of hydrosulphuric water. They were numbered: No 1—water with Eh=−170 mV, SCE, pH=6.6 and No 2—water with Eh=−20 mV, SCE, pH=8.7.

Part of solutions was used for creation of control samples with the Adler sludge. Natural Adler sludge is characterized by acid reaction (pH=5.7) and positive Eh=+438 mV, SCE. After mixing of sludge with hydrosulphuric water with characteristics, specified above, redox properties of raw material-containing water were sharply changed, in particular, sludge. Raw material-containing water has obtained redox properties, which are characterized by the redox potential Eh=−114 mV, SCE, and pH=6.6; Eh=−15 mV, SCE, and pH=7.8. Control samples were numbered accordingly No 2 and No 3.

So the samples No 1 and No 2 were the control samples of hydrosulphuric waters and No 3 and No 4 were the control samples of Adler mud/peloid with hydrogen sulfide. In the samples No 2 and No 4 concentration of hydrogen sulfide was about 12-20 mg per 100 ml of solution, and in the samples No 1 and No 3-350 mg per 100 ml of solution.

The samples were sealed and kept for testing in comparison with the experimental samples with neat sequence numbers:
No 5x—glycine with concentration of 0.005% of weight;
No 5—glycine with concentration of 0.5% of weight;
No 5f—glycine with concentration of 10% of weight;
No 6x—serine with concentration of 0.005% of weight;
No 6—serine with concentration of 0.5% of weight;
No 6f—serine with concentration of 10% of weight;
No 7x—threonine with concentration of 0.005% of weight;
No 7—threonine with concentration of 0.5% of weight;
No 7f—threonine with concentration of 10% of weight;
No 8x—cysteine with concentration of 0.005% of weight;
No 8—cysteine with concentration of 0.5% of weight;
No 8f—cysteine with concentration of 10% of weight;
No 9x—tyrosine with concentration of 0.005% of weight;
No 9—tyrosine with concentration of 0.5% of weight;
No 9f—tyrosine with concentration of 10% of weight;
No 10x—asparagine with concentration of 0.005% of weight;
No 10—asparagine with concentration of 0.5% of weight;
No 10f—asparagine with concentration of 10% of weight;
No 11x—glutamine with concentration of 0.005% of weight;
No 11—glutamine with concentration of 0.5% of weight;
No 11 f—glutamine with concentration of 10% of weight;
No 12x—glycine+cysteine with total concentration of reagents 0.005% of weight;
No 12—glycine+cysteine with total concentration of reagents 0.5% of weight;
No 12f—glycine+cysteine with total concentration of reagents 10% of weight;
No 13x—tyrosine+glutamine with total concentration of reagents 0.005% of weight;
No 13—tyrosine+glutamine with total concentration of reagents 0.5% of weight;
No 13f—tyrosine+glutamine with total concentration of reagents 10% of weight;
No 14x—serine+asparagine with total concentration of reagents 0.005% of weight;
No 14—serine+asparagine with total concentration of reagents 0.5% of weight;
No 14f—serine+asparagine with total concentration of reagents 10% of weight;
No 15x—threonine+serine+glutamine+glycine with total concentration of reagents 0.005% of weight;
No 15—threonine+serine+glutamine+glycine with total concentration of reagents 0.5% of weight;
No 15f—threonine+serine+glutamine+glycine with total concentration of reagents 10% of weight;

Production of the composition, corresponding to this invention, could be executed within the wide range of concentrations of ingredients: from 0.005% of weight to 10% of weight, 20% of weight and more. The range is limited by solubility of amino-acids, used according to this invention, in water, for example, hydrosulphuric solution. Optimal concentrations are established in order that the composition, corresponding to this invention, could provide the technologically required preservation of water solutions and raw material-containing water and was commercially available.

It was found out that the concentration of ingredients equal to 0.005% of weight, used in accordance with this invention, is the minimum, required for ensuring of preservation of water solutions and raw material-containing water with specified redox properties.

Biological activity of the composition, corresponding to this invention, is determined through the rate of influence of the mentioned composition on skin within different time intervals, which shows itself in hyperemia of skin in presence of active form of hydrogen sulfide in the composition, corresponding to this invention.

Tests were conducted in the following way:

Hydrosulphuric water from the sample No 1 was applied to the skin of thigh by sponge immediately after its preparation. Within 3-5 minutes, under influence of solution of hydrogen sulfide with concentration 340-370 mg/100 ml of solution, the skin becomes reddish, which means the skin hyperemia. 12 hours later hydrosulphuric water from the sample No 1 was applied by sponge once more to the skin of thigh of the same patient. Neither after 5 minutes, nor after 120 minutes there was no characteristic reaction of reddening of skin. In order to obtain data, which characterize redox properties of the sample No 1, there was used the pH-meter-millivoltmeter 1-120, the action of which is based on measurement of electromotive force of pair, which consists of the platinum electrode and auxiliary half-cell, in particular, silver chloride electrode, which are in contact with the water solution or peloid. In such conditions the potential of the platinum electrode depends on the rate of oxidation or reduction of reversible redox systems, for example, H₂S⇄HS−+H+. The value of Eh is determined as the algebraic sum of the measured potential and the potential of the silver chloride electrode of comparison (half-cell). Usually the value of Eh is expressed in millivolts or conditional units rH₂, where

$${rH}_{2} = \frac{Eh}{0.029} + 2 pH ( at temperature 18 ° C . ) .$$

In presence of reduction properties the redox potential Eh usually expressed by the negative value. The higher the biological activity of the hydrogen sulfide solution or peloid, the lower the value of redox potential. Measurement of potential for the sample No 1 gives the value Eh =+200 mV, SCE, while the initial value was Eh=−170 mV, SCE and pH=6.6. Specific odor of hydrogen sulfide was also not fixed. So after 12 hours the sample No 1 has lost balneological reaction and changed redox potential from negative to positive value in regard to the initial level. The examination of balneological reaction of the sample No 2 (experiment) 10 minutes after preparation has shown the presence of such reaction. For the group of the samples No 5-No 15 of water solution and raw material-containing water, saturated with hydrogen sulfide, with amino-acids with not charged polar substitutes in structure of amino-acids, including glycine, serine, threonine, cysteine, tyrosine, asparagine, glutamine and their mixtures, additionally added to the solution, there were obtained the following results. During examination of Eh and reaction of skin on the solution after 12 and 720 hours it was found out the increasing of Eh from −170 mV, SCE, to −160 (−110) mV, SCE, and presence of skin reaction on the solution both after 12 hours and after 720 hours.

Together with the experimental solutions with hydrogen sulfide there were prepared experimental samples of raw material-containing water which includes the Adler mud, saturated with hydrogen sulfide, with Eh=−114 mV and pH=6.6 with addition into this raw material of amino-acids in accordance with this invention, in particular, glycine, serine, threonine, cysteine, tyrosine, asparagine, glutamine and their mixtures. The samples were numbered from No 5a to No 15a. Concentration of organic substances in each of them was 0.5% of weight as the optimal for keeping mentioned properties of raw material-containing water. Tests for these samples were conducted in the same way as for samples No 5, 5x, 5f-No 15, 15x, 15f. Comparison was executed with control sample No 3 with characteristics: Eh=−114 mV, SCE, pH=6.6.

Control sample caused no balneological reaction of skin reddening after application to the skin of thigh of a patient after 12 hours (sample No 3). It was also no hydrogen sulfide odor. Measurements has registered the redox potential Eh=+270 mV, SCE. It gives the reason for assumption, that hydrogen sulfide in the sample No 3 was oxidized to SO, S⁰, SO₂ and HS−, which have a weak physiological influence on human organism. In experimental samples after 720 hours (one month) it was found out increasing of redox potential up to Eh=−80 mV, SCE, pH=6.6. There took place balneological reaction of skin reddening after application of peloids from samples No 5a-No 15a analogous to reaction of organism on application of water solutions of samples No 5, 5x, 5f-No 15, 15x, 15f.

TABLE 18
Stabilization of hydrosulphuric water by
amino-acids, specified in this invention

| | | Eh | | Skin | Skin | Skin |
|---|---|---|---|---|---|---|
| | Eh | after | Eh | reaction | reaction | reaction |
| | after | 12 | after | after | after | after |
| No | 10 min. | hours | 720 hours | 10 min. | 12 hours | 720 hours |
| 1 | −170 | +200 | +280 | + | − | − |
| (control) | | | | | | |
| 2 | −20 | +200 | +320 | + | − | − |
| (control) | | | | | | |
| 5 | −170 | −170 | −150 | + | + | + |
| 5x | −170 | −170 | −150 | + | + | + |
| 5f | −170 | −170 | −140 | + | + | + |
| 6 | −170 | −170 | −120 | + | + | + |
| 6x | −170 | −170 | −110 | + | + | + |
| 6f | −170 | −170 | −130 | + | + | + |
| 7 | −170 | −170 | −165 | + | + | + |
| 7x | −170 | −170 | −140 | + | + | + |
| 7f | −170 | −170 | −130 | + | + | + |
| 8 | −170 | −170 | −150 | + | + | + |
| 8x | −170 | −170 | −160 | + | + | + |
| 8f | −170 | −170 | −170 | + | + | + |
| 9 | −170 | −170 | −150 | + | + | + |
| 9x | −170 | −170 | −130 | + | + | + |
| 9f | −170 | −170 | −110 | + | + | + |
| 10 | −170 | −170 | −140 | + | + | + |
| 10x | −170 | −170 | −130 | + | + | + |
| 10f | −170 | −170 | −140 | + | + | + |
| 11 | −170 | −170 | −135 | + | + | + |
| 11x | −170 | −170 | −130 | + | + | + |
| 11f | −170 | −170 | −120 | + | + | + |
| 12 | −170 | −170 | −140 | + | + | + |
| 12x | −170 | −170 | −140 | + | + | + |
| 12f | −170 | −170 | −140 | + | + | + |
| 13 | −170 | −170 | −160 | + | + | + |
| 13x | −170 | −170 | −160 | + | + | + |
| 13f | −170 | −170 | −160 | + | + | + |
| 14 | −170 | −170 | −150 | + | + | + |
| 14x | −170 | −170 | −150 | + | + | + |
| 14f | −170 | −170 | −140 | + | + | + |
| 15 | −170 | −170 | −130 | + | + | + |
| 15x | −170 | −170 | −140 | + | + | + |
| 15f | −170 | −170 | −150 | + | + | + |

TABLE 19
Stabilization of hydrosulphuric mud by
amino-acids, specified in this invention

| | Eh | Eh | Eh | Skin | Skin | Skin |
|---|---|---|---|---|---|---|
| | after | after | after | reaction | reaction | reaction |
| | 10 | 12 | 720 | after | after 12 | after 720 |
| No | min. | hours | hours | 10 min. | hours | hours |
| 3 (control) | −114 | +270 | +310 | + | − | − |
| 4 (control) | −114 | +220 | +260 | + | − | − |
| 5a glycine | −114 | −114 | −100 | + | + | + |
| 6a serine | −114 | −114 | −90 | + | + | + |
| 7a threonine | −114 | −114 | −105 | + | + | + |
| 8a cysteine | −114 | −114 | −85 | + | + | + |
| 9a tyrosine | −114 | −114 | −90 | + | + | + |
| 10a | −114 | −114 | −85 | + | + | + |
| asparagine | | | | | | |
| 11a | −114 | −114 | −100 | + | + | + |
| glutamine | | | | | | |
| 12a glycine | −114 | −114 | −90 | + | + | + |
| cysteine | | | | | | |
| 13a tyrosine | −114 | −114 | −90 | + | + | + |
| glutamine | | | | | | |
| 14a serine | −114 | −114 | −90 | + | + | + |
| asparagine | | | | | | |
| 15a serine | −114 | −114 | −80 | + | + | + |
| threonine | | | | | | |
| glutamine | | | | | | |
| glycine | | | | | | |

### EXAMPLE 13

The Example of Production of the Composition, Corresponding to this Invention, from Water, Obtained by Contactless Method by Dissolution of Biologically Active Additive “Microhydrine”.

Experiment 1.

a) Control.

Widespread food additive “Microhydrine”, known by its antioxidant properties, as well as electrochemically (cathode) reduced water, has spontaneously changing redox properties, which are characterized by increasing of redox potential in relation to the potential of hydrogen electrode, the value of which is considered as zero. There were conducted experiments for obtaining the composition with stabilized redox properties in the basis of biologically active additive “Microhydrine”. Hermetically sealed thin-walled polyethylene container (thickness of walls is about 25 mcm) filled with distillated water (50 ml) was put into container of bigger volume (500 ml), also filled with distillated water. Then “Microhydrine” powder was added to the water of 500 ml volume and dissolved. Obtained water solution of “Microhydrine” has quickly possessed reduction properties, i.e. redox potential of obtained solution has decreased down to Eh=−500 mV, SCE with pH=8.7 and possessed the ability to contactless interaction with water in container with volume of 50 ml. After the maximal decreasing of the redox potential in the big container the 50 ml container was got out of the big one. Distillated water in 50 ml container has possessed the reduction properties: Eh=−370 mV, SCE, i.e. the redox potential of water in 50 ml container has decreased. Within 5 hours Eh of the water in 50 ml container has returned to the initial value. Conductivity of water during the experiment did not change. Changes in the solution of “Microhydrine” continued mach longer and not in usual way. The big container was sealed hermetically. During the first 7 days Eh gradually and smoothly increased up to Eh=−140 mV. During next 20 days the average value of Eh continued to grow up to Eh=+60 mV. During next 10 days Eh has increased to +240 mV without further changing. So the time of complete return of “Microhydrine” solution to water condition (in respect to redox potential) before addition of “Microhydrine” was 37 days.

b) Experiment.

Distillated water, which was intended for filling of the thin-walled polyethylene 50 ml container with wall thickness about 25 mcm, was mixed with glycine in concentration of 0.5% of weight. It does not exclude the use of other amino-acids according to this invention.

Hermetic thin-walled polyethylene container (wall thickness is about 25 mcm) with solution of glycine in 50 ml of distillated water was put into the bigger container (500 ml) filled with distillated water. Then “Microhydrine” powder was added to this water in the same amount as in the control experiment and agitated. Water solution of “Microhydrine” has quickly possessed spontaneously changing redox properties, which are characterized by spontaneous increasing of redox potential in relation to the potential of the hydrogen electrode, the value of which is considered as zero, with initial value of Eh=−500 mV, SCE, and has possessed the ability to contactless interaction with water in container with volume of 50 ml. When decreasing of Eh in the big container has stopped, the small one was got out of “Microhydrine” solution. Eh of water solution of glycine in 50 ml container has decreased down to Eh=−370 mV, SCE, i.e. it has possessed the reduction ability. During 6 months Eh of the water from small container has not returned to the initial value. In particular, during this time the redox potential has increased by 20% and pH has decreased from 8.7 to 7.05.

TABLE 20
Production of the composition, corresponding to this invention, by the
contactless method with use of the food additive “Microhydrine”

| | 1 day | 7 days | 20 days | 37 days | 2 months | 4 months | 6 months |
|---|---|---|---|---|---|---|---|
| Eh (control) | −500 mV | −140 mV | +60 mV | +240 mV | +240 mV | +240 mV | +240 mV |
| PH | 8.7 | 8.5 | 7.6 | 7.2 | 7.2 | 7.2 | 7.2 |
| Eh | −500 mV | −500 mV | −500 mV | −500 mV | −500 mV | −480 mV | −400 mV |
| (experiment) | | | | | | | |
| PH | 7.3 | 7.3 | 7.3 | 7.3 | 7.3 | 7.3 | 7.05 |

Experiment 2.
a) Control.

Hermetic, thin-walled (less than 0.1 mm) closed containers of dielectric material (ampoules or capsules) or tube made of polyvinyl chloride with physiological salt solution, containers, made of chemically inert, non-porous and non-conductive materials, were put into cathode reduced water, p0repared just before dipping of containers with the physiological salt solution. After 2 hours exposition of hermetic ampoules or tubes with the physiological salt solution in cathode (electrochemically) reduced water Eh and pH of physiological salt solution were significantly changed. It could be considered as possession by water solution in ampoules and tubes of specified redox properties by contactless interaction of physiological salt solution through the surface of these containers (ampoules, tubes) made of chemically inert, non-porous and not conductive material with electrochemically (cathode) reduces water solution with spontaneously changing redox properties. Two hours later pH and Eh, changed in result of contactless interaction, were transformed. In particular, pH has changed to 7.0, and Eh has reached the initial level during 5 hours.

b) Experiment.

Hermetic, thin-walled (less than 0.1 mm) closed containers of dielectric material (ampoules or capsules) or tube made of polyvinyl chloride with physiological salt solution, containers, made of chemically inert, non-porous and non-conductive materials, were put into electrochemically (cathode) reduced water with addition of glycine in concentration 0.5% prepared just before dipping of containers with the physiological salt solution. After 2 hours exposition of hermetic ampoules or tubes with the physiological salt solution in cathode (electrochemically) reduced water Eh and pH of physiological salt solution were significantly changed. It could be considered as possession by water solution in ampoules and tubes of specified redox properties by contactless interaction of physiological salt solution through the surface of these containers (ampoules, tubes) made of chemically inert, non-porous and not conductive material with electrochemically (cathode) reduces water solution with spontaneously changing redox properties. In opposite to the control, even 6 months later pH and Eh, changed in result of contactless interaction, were not changed so significantly, as in control experiment. In particular, pH has decreased down to pH=7.0, and Eh in the end of the 5^{th} month has increased only by 15%.

TABLE 21
Production of the composition, corresponding to this invention,
by the contactless method with use of the cathode reduced water

| | 2 hours | 5 hours | 1 month | 3 months | 6 months |
|---|---|---|---|---|---|
| Eh (control) | −80 mV | +170 mV | +230 | +210 | +240 |
| pH (control) | 7.9 | 7.2 | 7.2 | 6.9 | 6.7 |
| Eh | −500 mV | −500 mV | −500 mV | −470 mV | −430 mV |
| (experiment) | | | | | |
| pH | 7.3 | 7.4 | 7.1 | 7.2 | 7.3 |
| (experiment) | | | | | |

### EXAMPLE 14

Example, Demonstrating the Influence of the Composition with Stabilized Redox Properties on the System of Blood Coagulation.

The aim of this investigation is estimation of possible influence of the specified composition with redox potential Eh=−300 mV, SCE on the hemostasis system.

For this purpose influence of specified composition on some integral parameters of the blood coagulation system were investigated. Experiments were conducted on rabbits of both sexes with weight about 3.0-4.0 kg. All animals (n=12) were divided in two equal groups. For rabbits of the first group the specified composition was injected hypodermically in dose of 7.0 ml of initial substance per 1 kg of animal's weight during 14 days. For rabbits of the second (control) group there was injected ordinary boiled and cooled water in the same dose. Rabbit's blood was taken from ear's edge vein by the method of free drops' fall before the beginning of the experiment and 1 hour, 7 days and 14 days after the first injection of the specified composition.

For preparation of plasma rich in thrombocytes, blood was centrifuged during 10 minutes at speed of 1000 rpm, after which the upper plasma layer was transferred to another test tube, and remaining part was centrifuged once more during 20 minutes at speed of 3000 rpm for obtaining plasma, poor in thrombocytes.

There were investigated ADF-induced aggregation of thrombocytes, determined the number of thrombocytes, and activated partial time of coagulation (APTC), measured amount of fibrinogen and level of products of degradation of fibrin and fibrinogen (PDF), as well as activity of plasminogen activator of plasma type (t-PA).

Aggregation of thrombocytes was investigated by method of G. G. Born (1962) with aggregometer of the firm “Chrono-Long Corporation” (USA). ADF in final concentration of 1×10⁵ M were used as pro-aggregants. For this purpose 450 mkl of plasma, rich in thrombocytes, were poured into the tray of the instrument; the same volume of plasma without thrombocytes was used for optical control. The degree of aggregation was determined through maximal value of optical density drop after the end or the reaction (Aₘₐₓ) in comparison with the initial value. ADF in final concentration of 1×10⁵ M were used as pro-aggregants.

Number of thrombocytes was measured by optical method. Initial number of thrombocytes was assumed as 100%. There was also was determined the amount of fibrinogen with use of coagulometer, determined fibrin and fibrinogen degradation products with use of the sets “Fibro-Tec”. This method is based on ability of cellular membranes to form precipitate, which is visible without use of special instruments.

In process of analysis of 2-weeks injections of the specified composition with Eh=−300 mV, SCE, on coagulological blood potential there was found out that during period of experiment there was not observed any changes in ADF-induced aggregation of thrombocytes, prothrombin time, activated partial thromboplastin time and content of plasminogen in the rabbit plasma. Amount of fibrin and fibrinogen degradation products both before the experiment and during investigation did not exceed the physiological norm (tables 22-28).

TABLE 22
Influence of the composition, corresponding to this invention,
on the value of activated partial thromboplastin time (sec)

| | Before | After first injection | | |
|---|---|---|---|---|
| | injection | 1 hour | 7 days | 14 days |
| Control | 17.1 | 17.5 | 18.9 | 19.0 |
| Experiment | 19.2 | 19.5 | 18.9 | 19.5 |

TABLE 23
Influence of composition, corresponding to this invention
on the value of the prothrombin time (sec)

| | Before | After first injection | | |
|---|---|---|---|---|
| | injection | 1 hour | 7 days | 14 days |
| Control | 8.5 | 9.1 | 8.1 | 8.0 |
| Experiment | 8.0 | 8.2 | 8.0 | 8.2 |

TABLE 24
Influence of composition, corresponding to this
invention on content of fibrinogen (g/l)

| | Before | After first injection | | |
|---|---|---|---|---|
| | injection | 1 hour | 7 days | 14 days |
| Control | 3.7 | 3.8 | 4.4 | 4.2 |
| Experiment | 4.6 | 4.3 | 4.6 | 4.6 |

TABLE 25
Influence of the composition, corresponding to this invention on
aggregation of thrombocytes (ADF 1 × 10⁵ M; Aₘₐₓ; %)

| | Before | After first injection | | |
|---|---|---|---|---|
| | injection | 1 hour | 7 days | 14 days |
| Control | 39.0 | 43.0 | 39.0 | 37.0 |
| Experiment | 41.1 | 41.0 | 39.0 | 42.0 |

TABLE 26
Influence of the composition, corresponding to this invention on
the level of fibrin and fibrinogen degradation products (mkg/ml)

| | Before | After first injection | | |
|---|---|---|---|---|
| | injection | 1 hour | 7 days | 14 days |
| Control | 0 | 0 | 0 | 0 |
| Experiment | 0 | 0 | 0-0.6 | 0 |

Comparative investigation of influence of the specified composition and ordinary water on the number of thrombocytes has revealed that, while the specified composition does not cause change of this characteristic, hypodermic injection of ordinary water in investigated dose caused increase of the number of thrombocytes after 1 hour after beginning of the experiment, and this effect was stable till the end of the experiment.

TABLE 27
Influence of the composition, corresponding to this invention (Eh =
−300 mV, SCE), on the number of thrombocytes (% of initial level)

| | Before | After first injection | | |
|---|---|---|---|---|
| | injection | 1 hour | 7 days | 14 days |
| Control | 100 | 140* | 177* | 153* |
| Experiment | 100 | 108** | 102 8** | 100** |

*reliable in relation to initial level (p < 0.05)
**reliable in regard to the number of thrombocytes in the corresponding control group (p < 0.05).

Determination of activity of plasminogen activator of tissue type in the control and the experimental groups has shown that, while the injection of ordinary water was accompanied by reliable increase of activity t-PA 7 and 14 days after the beginning of the experiment, hypodermic injection of the specified composition caused increase of this characteristic 1 hour after beginning of the experiment, and after 7 and 14 days activity of t-PA does not exceed the initial level.

TABLE 28
Influence of the composition, corresponding to this invention
on activity of plasminogen activator of tissue type (%)

| | Before | After first injection | | |
|---|---|---|---|---|
| | injection | 1 hour | 7 days | 14 days |
| Control | 97.0 | 121.0* | 122.0* | 134.0* |
| Experiment | 103.0 | 146.0** | 98.0** | 85.0** |

*reliable in relation to initial level (p < 0.05)
**reliable in regard to the number of thrombocytes in the corresponding control group (p < 0.05).

Analysis of obtained results allowed to conclude that the specified composition with redox potential Eh=−300 mV, SCE, in case of hypodermic injection to rabbits in dose of 7.0 ml per kg of animal's weight did not cause essential changes of blood coagulological potential. There is shown the absence of influence of the specified composition on functional condition both of internal way of activation of hemostasis (no changes of APTC value), and of external way, which is confirmed by stability of prothrombin time value during experiment. The specified composition also did not cause initiation of disseminated intravascular blood coagulation, which is confirmed by absence of changes in content of fibrinogen, PDF and number of thombocytes. Thrombocytosis, developed as response on injection of ordinary water is probably determined by cells' exit from depot in response on increase of circulating blood volume. The specified composition did not cause such changes, which could be one of the reasons of known positive effect of so called “ionized water” in case of some conditions, connected with disturbance of vascular tension regulation.

So, under influence of the composition with stabilized redox properties (experiment) there takes place the increase of activity of plasminogen activator of tissue type after its the first injection, and then begins adaptation to the water load. In case of hypodermic injection of ordinary water the increase of t-PA activity takes place during the experiment, and it indicates the absence of protective reaction on the sharp change of circulating liquid volume.

So the composition, corresponding to this invention, with Eh=−300 mV, SCE, has no negative influence on the hemostasis system.

### EXAMPLE 15

The Example, Demonstrating the Influence of the Composition with Stabilized Redox Properties on the Process of Healing of Wounds.

The influence of the specified composition on the process of healing of wounds was investigated in experiment on 20 white rats. The composition, corresponding to this invention, was the specified composition with pH=9±0.4, Eh=−300 mV and mineralization up to 0.2 g/l. Plain skin wounds were made to rats on back by special puncher without observation of sterile conditions. There were formed two skin defects with diameter 10 mm. Anesthesia was made by intra-abdominal administration of 0.1% solution of hexenal. Skin defects were opened during period of observation (10 days). In experimental series wounds were irrigated two times a day by the mentioned composition, in control series—by distillated water. Every two days there were examined clinical conditions of wounds, determined their dimensions, carried out bacteriological investigations. After the end of experiment on the 10^{th} day there was mad the sampling of tissues from the wound area and from surrounding areas of non-damaged skin for histological analysis. Wounds in control group during the first 5 days were covered by wet crust with light-yellow discharge without odor. There were bright ridges around wounds, indicating expressed process of traumatic inflammation. In result the dimensions of wounds exceeded the initial one—11.6±0.4 mm. During next 5 days the surface of wounds was decreased by 50-60% due to contraction and border epithelization. Two rats from ten had expressed purulent complications. Bacteriological investigations have shown that during first 5 days dissemination of wound is 770-840 colonies of Staphylococcus aureus. Then after forming of the hard crust and beginning of the border epithelization dissemination is increased down to 360-300 colonies of microorganisms. On 10^{th} day the complete healing was observed only for one animal, other have wounds with dimensions 5-6 mm in diameter with signs of inflammation.

For animals, wounds of which were treated by the specified composition, the process of healing during the first 2-3 days was developed in the other way in comparison with the control group. Dimensions of wounds were decreased by 15-20%, inflammation process was expressed in a lesser degree in comparison with the control series, dissemination of wounds was 300-370 colonies of Staphylococcus aureus. On 5^{th} and especially on 7^{th} day there was observed the sharp acceleration of healing process. On 10^{th} day the complete healing was observed for 4 rats, other had small defects (2-3 mm in diameter), covered with dry crust.

The use of the specified composition is effective both for the first and for the second and third phase of wound process, on the stage of proliferation of fibroblasts and growth of vessels, fibrillogenesis of collagen, ripening and fibrous transformation of granular tissue, reorganization of cicatrix.

### EXAMPLE 16

Example of Including of the Water Solution, Containing Glycine, with the Specified Redox Properties, in Content of Liposomes for Creation of the Composition, Corresponding to this Invention, for Use in Cosmetology.

In the experiment liposomes are made by mixing and ultrasound treatment of phospholipids of yolk and the water solution, corresponding to this invention, with specified redox properties, containing glycine. Liposomes, prepared by this method, are the milk-white suspension. Water volume of liposomes changes from 1 to 4 litres per mole of mixture of phospholipids and depends both on conditions of preparation (temperature, time, intensity of mixing, nature of phospholipids) and on redox potential (Eh) and pH of water phase. It was found out that liposomes with included in their content water solution with specified redox properties, containing glycine in concentration of 0.5%, could be kept not less than 6 months with Eh from −150 mV to −300 mV, SCE, mineralization up to 0.2 g/l and pH from 5.5 to 7.5. It was found out earlier that the cathode-reduced water or catholyte, included into micro-capsules (liposomes) is the universal stimulator of cell metabolism, stabilizes cell membranes, decelerates skin ageing and is used in composition of cosmetic raw materials with taking into account that the catholyte used in mentioned cream is characterizes by mineralization about 9 g/l, Eh=−500 mV, SCE, and pH>9, which is the border of physiological values (see, for example, V. I. Prilutsky, V. M. Bakhir. Electrochemically activated water: anomalous properties, mechanism of biological effect. Moscow 1997, VNIIIMT, p. 152).

There was also determined the rate of oxidation of phospholipids according to content of dialdehyde malonate (DMA).

Table 29 presents the results of determination of DMA (nmole/ml) in different liposome dispersions.

TABLE 29
Content of DMA in liposomes dispersions

| | | |
|---|---|---|
| | | Content of MDA |
| Dispersion medium | Time | (nmole/ml |

Yolk lecithin

| | | |
|---|---|---|
| Water | 1 hour | 2.5 |
| Composition, corresponding to this | 1 hour | 2.2 |
| invention | | |
| Water + tocopherol acetate (2%) | 1 hour | 3.1 |
| Water + butyl-hydroxytoluene (0.1%) | 1 hour | 2.7 |
| Water + agidol (1.0%) | 1 hour | 2.0 |
| Water | 1 day | 11.0 |
| Composition, corresponding to this | 1 day | 6.9 |
| invention | | |
| Composition, corresponding to this | 2 weeks | 3.2 |
| invention | | |
| Water | 2 weeks | 14.2 |

Conclusion: the offered composition, corresponding to the invention, more than four times decelerates the rate of oxidation of phospholipids in liposomes, which is unobtainable for known used methods, taking into account the absence of antioxidants, preventing oxidation and at the same time causing no phase separation in liposome dispersion.

### Industrial Applicability

There is presented the composition with stabilized redox properties, which corresponds to water solution or raw material-containing water with spontaneously changing redox properties, which are characterized by spontaneously increasing of the redox potential in relation to the potential of the hydrogen electrode, the value of which is considered as zero, in which the redox properties are stabilized by addition of amino-acids with not charged polar substitutes and/or their derivatives, and/or peptides, containing specified amino-acids and/or their derivatives, and/or their mixtures. This composition could be used in food industry, medicine, veterinary, pharmaceutical industry, cosmetic industry, balneology, agriculture, fish farming and other fields.

1. The composition with stabilized redox properties, which corresponds to water solution and/or raw material-containing water with spontaneously changing redox properties, which are characterized by spontaneous increasing of the redox potential in relation to the potential of the hydrogen electrode, which value is considered as zero, in which redox properties are stabilized by addition of amino-acids with not charged polar substitutes and/or their derivatives and/or peptides, containing mentioned amino-acids and/or their derivatives and/or their mixtures.

2. The composition according to the paragraph 1, in which the mentioned amino-acids are represented by glycine, serine, threonine, cysteine, tyrosine, asparagine or glutamine.

3. The composition according to the paragraph 1, in which the mentioned peptide is represented by gelatin.

4. The composition according to the paragraph 1, in which the amount of amino-acids with not charged polar substitutes and/or their derivatives and/or peptides, containing mentioned amino-acids and/or their derivatives and/or their mixtures, is more than 0.005% of weight.

5. The composition according to the paragraph 1, in which the water solution and/or raw material-containing water is represented by foodstuff, including mineral and/or drinking water, milk composition, juice, alcoholic and/or alcohol-free drink, mayonnaise, ketchup, sauce, meat, fish, vegetable and/or fruit semi-manufactured product, sausage and/or canned composition, confectionery, bread, macaroni, foodstuff with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

6. The composition according to the paragraph 1, in which the water solution and/or raw material-containing water is represented by the balneological composition, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

7. The composition according to the paragraph 1, in which the water solution and/or raw material-containing water is represented by the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive and/or vitamin mixture, liquid for contact lenses, eye drops, basis for medical preparation, influencing different kinds of metabolism, including carbohydrate metabolism, phosphoric and calcium metabolism, homeostasis, hemopoiesis, hemostasis; agent, influencing immunity, correcting anti-tumoural therapy, antibiotics therapy, radio therapy used in gynecology, otorhynolaryngology, dentistry, ophthalmology, proctology, urology, for external use, dermatology; agents with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

8. The composition according to the paragraph 1, in which the water solution and/or raw material-containing water is represented by the composition for cosmetic purpose, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

9. The composition according to the paragraph 1, in which the water solution and/or raw material-containing water is represented by the composition for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

10. The composition according to the paragraph 1, in which the water solution and/or raw material-containing water is represented by the composition for veterinary purposes, including feed and drink and/or medical preparation with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

11. The composition according to the paragraph 1, in which the water solution and/or raw material-containing water, which could be used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.

12. The method of stabilization of redox properties of composition, which corresponds to water solution and/or raw material-containing water with spontaneously changing redox properties, which are characterized by spontaneous increasing of the redox potential in relation to the potential of the hydrogen electrode, which value is considered as zero, which consists in addition of amino-acids with not charged polar substitutes and/or their derivatives and/or peptides, containing mentioned amino-acids and/or their derivatives and/or their mixtures.

13. The method according to the paragraph 12, in which the mentioned amino-acids are represented by glycine, serine, threonine, cysteine, tyrosine, asparagine or glutamine.

14. The method according to the paragraph 12, in which the mentioned peptide is represented by gelatin.

15. The method according to the paragraph 12, in which the amount of amino-acids with not charged polar substitutes and/or their derivatives and/or peptides, containing mentioned amino-acids and/or their derivatives and/or their mixtures is more than 0.005% of weight.

16. The method according to the paragraph 12, in which the water solution and/or raw material-containing water is represented by foodstuff, including mineral and/or drinking water, milk composition, juice, alcoholic and/or alcohol-free drink, mayonnaise, ketchup, sauce, meat, fish, vegetable and/or fruit semi-manufactured product, sausage and/or canned composition, confectionery, bread, macaroni, foodstuff with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

17. The method according to the paragraph 12, in which the water solution and/or raw material-containing water is represented by the balneological composition, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

18. The method according to the paragraph 12, in which the water solution and/or raw material-containing water is represented by the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive and/or vitamin mixture, liquid for contact lenses, eye drops, basis for medical preparation, influencing different kinds of metabolism, including carbohydrate metabolism, phosphoric and calcium metabolism, homeostasis, hemopoiesis, hemostasis; agent, influencing immunity, correcting anti-tumoural therapy, antibiotics therapy, radio therapy used in gynecology, otorhynolaryngology, dentistry, ophthalmology, proctology, urology, for external use, dermatology; agents with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

19. The method according to the paragraph 12, in which the water solution and/or raw material-containing water is represented by the composition for cosmetic purpose, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

20. The method according to the paragraph 12, in which the water solution and/or raw material-containing water is represented by the composition for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

21. The method according to the paragraph 12, in which the water solution and/or raw material-containing water is represented by the composition for veterinary purposes, including feed and drink and/or medical preparation with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

22. The method according to the paragraph 12, in which the water solution and/or raw material-containing water, which could be used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.

23. The use of amino-acids with not charged polar substitutes and/or their derivatives and/or peptides, containing mentioned amino-acids and/or their derivatives and/or their mixtures for stabilization of redox properties of composition, which corresponds to water solution and/or raw material-containing water with spontaneously changing redox properties, which are characterized by spontaneous increasing of the redox potential in relation to the potential of the hydrogen electrode, which value is considered as zero.

24. The use according to the paragraph 22, in which the mentioned amino-acids are represented by glycine, serine, threonine, cysteine, tyrosine, asparagine or glutamine.

25. The use according to the paragraph 22, in which the mentioned peptide is represented by gelatin.

26. The use according to the paragraph 22, in which the amount of amino-acids with not charged polar substitutes and/or their derivatives and/or peptides, containing mentioned amino-acids and/or their derivatives and/or their mixtures is more than 0.005% of weight.

27. The use according to the paragraph 23, in which the water solution and/or raw material-containing water is represented by foodstuff, including mineral and/or drinking water, milk composition, juice, alcoholic and/or alcohol-free drink, mayonnaise, ketchup, sauce, meat, fish, vegetable and/or fruit semi-manufactured product, sausage and/or canned composition, confectionery, bread, macaroni, foodstuff with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

28. The use according to the paragraph 22, in which the water solution and/or raw material-containing water is represented by the balneological composition, including mineral water, mud (peloid), clay, peat, sludge, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

29. The use according to the paragraph 23, in which the water solution and/or raw material-containing water is represented by the composition for therapeutic and prophylactic purpose, including tooth elixir, paste, lotion, cream, water and/or oil extract of medicative herbs, biogenic preparation, gel, aerosol, tampon, deodorant, wet hygienic napkin, bandage, cotton, hydrogel tampon, collegan film, algiporic gel, coal, micro-crystal cellulose and/or polysaccharide sorbent, pectin, polyphepam, zeolite, chitin and/or chitosan film, gel, powder, solution, nutritious mask, shampoo, conditioner, solution for correction of electrolytic and/or acid-alkaline balance, solution for dialysis, nutritive and/or vitamin mixture, liquid for contact lenses, eye drops, basis for medical preparation, influencing different kinds of metabolism, including carbohydrate metabolism, phosphoric and calcium metabolism, homeostasis, hemopoiesis, hemostasis; agent, influencing immunity, correcting anti-tumoural therapy, antibiotics therapy, radio therapy used in gynecology, otorhynolaryngology, dentistry, ophthalmology, proctology, urology, for external use, dermatology; agents with disinfectant and/or antiseptic effect, preparation for treatment of disbacteriosis, antiphlogistic agent, antimicrobe agents for different groups, virulicide and antiviral agent, antituberculous agent, antimycotic agent, agent used in gastroenterology and/or in hepatology, bronchopulmonary agent, antiallergic, and also physiologic salt solution, parenteral agent for rehydration and/or detoxication, agent for correction of electrolytic and/or acid-alkaline balance, agent for parenteral alimentation, multivitamin agent with complex of biogenic adoptogens, amino-acid preparation, preparation using in case of functional asthenia, corrective food additive, plasma substituting and/or artificial blood substitutes, medical agent for external, intracavitary, intravenous, intramuscular, intraperitoneal, hypodermic, intradermal and/or internal administration, acting as disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

30. The use according to the paragraph 23, in which the water solution and/or raw material-containing water is represented by the composition for cosmetic purpose, including tooth paste, elixir, tampon, cream, gel, aerosol, perfume, eau-de-Cologne, lotion, deodorant, wet hygienic napkin, shampoo, conditioner, cosmetic agent with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

31. The use according to the paragraph 23, in which the water solution and/or raw material-containing water is represented by the composition for animal breeding, including medical preparation, feed and drink with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

32. The use according to the paragraph 23, in which the water solution and/or raw material-containing water is represented by the composition for veterinary purposes, including feed and drink and/or medical preparation with properties of disinfectant, antiseptic, preservation agent, antioxidant, antimutagen, radio-protector, immunostimulator, adoptogen, as virulicide, antiviral, antiphlogistic agent, as stimulator of regeneration of tissues and as stimulator of mitotic activity of enteric bacterial micro flora useful for man and/or animals.

33. The use according to the paragraph 23, in which the water solution and/or raw material-containing water, which could be used as fertilizer for agriculture with properties of disinfectant, antiseptic, preservation agent, antioxidant, antimutagen, radio-protector, immunostimulator, adoptogen, as virulicide, and antiviral agent, as stimulator of plants growth and as stimulator of mitotic activity of microbiologic flora useful for plants.
